(19) United States
(12) Patent Application Publication
(10) Pub. No.: US 2004/0209315 A1
(43) Pub. Date: Oct. 21, 2004

(54) Methods to identify agents that increase or decrease UBP43 activity and methods for use of such agents

(76) Inventors: Dong-Er Zhang, San Diego, CA (US); Ming Yan, San Diego, CA (US); Oxana A. Malakhova, San Diego, CA (US)

Correspondence Address: BAKER & BOTTS, 30 ROCKEFELLER PLAZA, NEW YORK, NY, 10112

(21) Appl. No.: 10/771,951

(22) Filed: Feb. 3, 2004

Related U.S. Application Data
(60) Provisional application No. 60/444,941, filed on Feb. 3, 2003.

(51) Int. Cl. G01N 33/53; G01N 33/567

(52) U.S. Cl. 435/7.2

(57) ABSTRACT

The present invention is directed to identification of agents that modulate UBP43 activity as well as associated methods, uses, processes, compositions and agents. In particular, the invention is directed to in vivo and in vitro methods to identify an agent that inhibits or stimulates UBP43 activity within a cell, a method for inducing cellular apoptosis, a method for affecting cellular reaction to interferon, a method for treating disease associated with cellular proliferation by causing apoptosis, and a method for treating both acute and chronic diseases in which interferon exerts a beneficial effect. The invention is also directed to modified ISG15-conjugates that have lowered or no susceptibility to UBP43 cleavage, pharmaceutical compositions of the agents, conjugates, and additional modified ISG15-conjugates.

### FIELD OF THE INVENTION

The invention relates generally to methods to identify inhibitors or activators of UPB43. The invention also relates to the use of inhibitors of UBP43 to treat, for example, cell-proliferative disorders, viral-related disorders, and autoimmune-related disorders. In addition, the invention relates to methods to prolong and increase the effects of interferon treatment of cells. Such methods may be used to treat disease related to viral infection, cellular proliferation, and autoimmune diseases such as multiple sclerosis.

### BACKGROUND OF THE INVENTION

The interferons (IFNs) comprise a family of multifunctional polypeptides, recognized for their antiviral, antiproliferative, and immunoregulatory functions. Type I IFNs, IFN-α and IFN-β exert their biological actions by binding to high-affinity cell-surface receptors that stimulate phosphorylation of tyrosine residues on type I receptor components and on the receptor-associated tyrosine kinases, TYK-2 and JAK-1. Following tyrosine phosphorylation, the activated tyrosine kinases induce the formation and activation by tyrosine phosphorylation of the latent cytoplasmic transcription factor, IFN-stimulated gene factor 3χ (ISGF3α). The ISGF3α transcription factor is a complex of STAT molecules, including STAT1 and STAT2. STAT1 exists in two forms, Stat1α (91 kDa) and STAT1β (84 kDa). STAT2 is thought to exist in one form (113-kDa). During the classical type I IFN response, the tyrosine-phosphorylated STAT1 and STAT2 proteins form heterodimers, which complex with p48-ISGF3γ, a DNA-binding protein. The ISGF3 complex then migrates to the nucleus and binds to the interferon stimulated response element (ISRE) to activate transcription of IFN-regulated genes.

The effect of IFN in the treatment of advanced malignant melanoma has been demonstrated in several clinical trials with an overall response rate of approximately 22%. In addition, analysis of results from in vitro studies using tumor colony-forming assays suggests that approximately 50% of the tumors contain cells that are nonresponsive to the antiproliferative effects of IFNs. The reasons for the differences in responsiveness of human melanoma cells to IFN in vitro and in vivo remain unclear. However, studies have reported various defects in the IFN system as being responsible for the different sensitivities to type 1 IFNs in cell lines established from other tumor types. Such defects include: i) IFN-α/IFN-β gene deletion in acute leukemia cell lines and malignant T cells; ii) alteration or down-regulation of IFN-α receptor gene expression in hairy cell leukemia and lymphoblastoid cells; iii) interference with the induction of the expression of IFN-stimulated genes in B lymphoid cell lines and Burkitt's lymphoma cells; iv) defects in the activation of transcription factors in Daudi cells, primary leukemia cells, and in many other cancer cell types. In addition, melanoma cell lines with a wide variation in their responsiveness to the antiproliferative and antiviral activities of IFNs have been described.

Accordingly, a need exists for methods and agents that can be used to increase the antiproliferative effectiveness of interferons as well as to cause hyperresponsiveness to interferons. Such methods and agents have great utility for use in the treatment of diseases sensitive to IFN therapy, diseases related to cellular proliferation, viral diseases, and autoimmune diseases.

### SUMMARY OF THE INVENTION

These and other needs are met by the present invention, which is directed to identification of agents that modulate UBP43 activity as well as associated methods, uses, processes, compositions and agents. In particular, embodiments, the present invention is directed to in vivo and in vitro methods to identify an agent that inhibits or stimulates UBP43 activity within a cell, a method for inducing or enhancing cellular apoptosis, a method for modulating cellular response to interferon, a method for treating diseases that may be responsive to interferon therapy, a method for treating diseases associated with cellular proliferation, and autoimmune-related diseases. The present invention is also directed to compositions comprising an ISG15-bound protein (“ISG15-conjugate”). The present invention is also directed to compositions comprising a modified ISG15-conjugate that has lowered or no susceptibility to UBP43 cleavage. The present invention is also directed to pharmaceutical compositions comprising an ISG15-conjugate.

The method for identifying agents for modulating UBP43 activity involves determining whether the agents stimulate, inhibit, increase and/or decrease the ability of the UBP43 enzyme (or variant thereof) to deconjugate ISG15 conjugates. The method may be conducted by in vitro or in vivo techniques. In one embodiment, the method generally involves incubating a reaction mixture containing a combination of an ISG15-conjugate that has ISG15 coupled to a target protein, and the deconjugation (decoupling) enzyme UBP43 under conditions designed to enable UBP43 to hydrolyze or otherwise decouple ISG15 and the target protein. After establishing control test results for deconjugation of an ISG15-conjugate in the absence of an agent, the agent of concern is added to the reaction mixture and deconjugation of the ISG15-conjugate to form free ISG15 and free target protein is determined. Comparison of test results with control results allows a determination whether the candidate agent induces, inhibits, decreases, increases and/or activates deconjugation. The rate of deconjugation, amount of ISG15 or target protein formed, redirection of the enzyme and other reaction parameters and/or results can be used to determine whether the agent alters deconjugation by UBP43. In a particular embodiment, the test can be conducted in vitro by appropriate combination in aqueous medium; in a preferred non-limiting embodiment, buffered isotonic medium. In another particular embodiment, the test can also be conducted in vivo by appropriate incubation in a cellular medium wherein the cells are engineered to express an ISG15-conjugate, the UBP43 enzyme (or variant thereof) and combinations thereof. The agent can be exogeneously combined.

The present invention is also directed to a method for inducing and/or enhancing cellular apoptosis. The method comprises administering to a patient, or contacting with a sample of cells, an agent that modulates the activity of the UBP43 enzyme, or a variant thereof. Although not being bound to any particular theory, negative modulation of UBP43 activity is believed to cause a build-up of ISG15-conjugate and a decrease of free target protein in cells, which leads to cellular apoptosis. It is also theorized that a homeostatic balance of free target protein and ISG15-conjugate is important for continued cellular growth and viability. When the enzymatic activity of UPB43 is reduced or inhibited, apoptosis and cellular death can occur. In accordance with the present invention, apoptosis is induced and/or enhanced by, for example, a method comprising administering to a patent in need of such treatment an agent that inhibits UPB43.

The present invention is also directed to a method for modulating cellular response to interferon. As mentioned above, UPB43 is believed to be involved in the homeostatic balance of target proteins needed for cellular growth and life. This balance is thought to involve a series of factors that include the ISG15 protein and the conjugation enzyme UBE1L. The cascade leading to the homeostatic balance can be modified by extracellular messengers such as interferon. Interferon stimulates ISG15 activity and is involved in a positive feedback pathway based upon UBP43. Hence, by altering the activity and/or concentration of UBP43, cellular sensitivity and response to interferon can be altered. Accordingly, the method of the invention involves formation of an in vivo test cell as described above, including the interferon receptor, ISG15 expression, target protein expressing UBE1L expression and UBP43 expression. After determining control test parameters and parameters involving an agent affecting UBP43 expression as described above, an interferon test is conducted. The cellular configuration is contacted with interferon in the presence and absence of the agent. The cellular response as described above will indicate whether there is increased sensitivity or otherwise altered response to interferon treatment.

The present invention also is directed to a method for diagnosing a patient having a malcondition characterized by an altered level of UBP43 enzyme or UBP43 enzyme activity. The method involves contacting a sample from a patient suspected of having such a malcondition of concern with a ISG15-conjugate and determining the ability of the sample to deconjugate the ISG15-conjugate. Using techniques known in the art, the ability to deconjugate the ISG-conjugate may be determined by rate study, determination of build-up of free target protein, determination of free ISG15 concentration, and the like. The rate, enzyme product concentrations, and other measurable parameters of enzymatic activity may be compared with similar parameters obtained using samples from a normal patient.

The comparison will show, for example, whether the UBP43 enzyme activity of the patient sample is higher or lower than normal. In one embodiment, if the enzyme activity is higher than normal, a shift in the homeostatic balance toward free target protein is indicated. This condition indicates increased cellular proliferation. In another embodiment, the opposite shift indicates increased cellular apoptosis.

The present invention is also directed to a method for treating a bacterial infection, a viral infection, a cell proliferative disorder, or an autoimmune disorder (e.g., multiple sclerosis). The method comprises administering an agent that appropriately alters the enzymatic activity of UBP43, for example, by stimulating, increasing, decreasing and/or inhibiting such activity.

The present invention also is directed to isolated ISG15-conjugates. In particular, non-limiting embodiments, the present invention is directed to modified ISG15-conjugates that have lowered or no susceptibility to UBP43 enzymatic cleavage when compared to respective unmodified ISG15-conjugates. In further non-limiting embodiments, such conjugates will comprise the ISG15 and target protein fragments, but in the region of conjugation will have epitopal sequences that strongly bind with the enzymatic pocket of UBP43. Such conjugates may be used as inhibitors of UBP43 enzymatic activity.

Pharmaceutical compositions comprising the above agents, modified ISG15-conjugates, and/or the like provide effective delivery formulations according to the invention.

### BRIEF DESCRIPTION OF FIGURES

FIG. 1 shows that UBP43−/− mice are hypersensitive to the injection of a potent IFN inducer—poly(I—C). (a) Seven mice of each group (UBP43 +/+ and UBP43−/−) were ip injected daily with poly(I—C). Viability was monitored daily. (b) UBP43+/+ and UBP43−/− mice (n=4 for each group) were ip injected daily with poly(I—C). The total number of white blood cells (WEC) in the peripheral blood was counted daily after each poly(I—C) injection. (c) UBP43+/+ and UBP43−/− mice (n=5 for each group) were either left untreated or were ip injected with poly(I—C) 24 h prior to bone marrow extraction. Bone marrow cells were counted in control and poly(I—C) injected group of mice (two femurs from each mouse).

FIG. 2 shows that the hypersensitivity to IFN stimulation is intrinsic in UBP43 deficient hematopoietic cells. (a) Hematopoietic cells transplanted from UBP43−/− mice to UBP43+/+ congenic strain mice are hypersensitive to poly(I—C) injection. Bone marrow cells (CD45.2+) from individual UBP43+/+ and UBP43−/− mice (n=4 for each group) were isolated and transplanted to lethally irradiated congenic strain mice (CD45.2−). Six months after the transplantation, flow cytometric analysis was done to determine the relative amount of peripheral blood cells contributed from the donor or recipient mice based on CD45-2 immunostaining before and after poly(I—C) injection (48 h). Flow cytometric analysis results from one representative mouse transplanted with UBP43+/+ or UBP43−/− bone marrow are presented. (b) UBP43−/− bone marrow cells are hypersensitive to IFNβ treatment. Bone marrow cells from UBP43+/+ and UBP43−/− mice were cultured at 2×10⁴ cells/ml in CFU assay medium in the presence of various concentrations of IFNβ. Total colony number was counted after 12 days of culture. Results of a single representative experiment of two independent experiments are shown. Data points represent mean (±SD) colony growth derived from colony counts in three replicate plates for each mouse with two mice in each group. (c) Increased level of ISG15 conjugation in UBP43−/− bone marrow cells. Bone marrow cells were either left untreated or stimulated with 100 unit/ml IFNβ for 24 hours. Total proteins (15 μg) were resolved on 6-18% gradient SDS-PAGE and immunoblotted with anti-mouse ISG15 antibodies (Abs). The bottom panel shows the membrane stained by Ponceau S.

FIG. 3 shows that the augmented apoptosis rate of UBP43−/− cells is specific to type I IFN treatment. (a) Bone marrow cells isolated from UBP43+/+ and UBP43−/− mice were cultured in vitro in the presence or the absence of INFβ (100 units/ml) for 48 hours. TUNEL assay was performed to study the percentage of apoptosis cells. (b) 48 hours after UBP43−/− bone marrow cells were infected with UBP43-MigR1, they were cultured in the presence or the absence of IFNβ (100 units/ml) for 48 hours. The cells (15% EGFR+ and 85% EGFR−) were stained with Annexing V-PE and 7-AAD and analyzed by flow cytometry. (c) Bone marrow cells from UBP43± and UBP43−/− mice were cultured in the absence or presence of 10 ng/ml IFγ, 10 ng/ml TNFα, and 100 units/ml IFNβ for 48 hours. Cells were then subjected to Trypan blue dye exclusion assay. Each data point represents the mean of three independent experiments. Error bar represents standard deviation about the mean.

FIG. 4 shows that the JAK-STAT pathway is extensively activated in UBP43−/− cells upon IFN stimulation. (a) ISFG3-complex formation in UBP43 deficient cells upon IFNβ stimulation. UBP43+/+ and UBP43−/− bone marrow cells were either left untreated or stimulated for the time indicated. Total protein extracts (30 μg) were used in the gel shift assays with ³²P-labeled double stranded oligonucleotide that contains an ISGF3 binding site. (b) UBP43+/+ and UBP43−/− bone marrow cells were either left untreated or stimulated with 100 unit/ml IFNβ as described above. Total proteins (15 μg) were resolved on 7% SDS-PAGE and immunoblotted with anti-phospho-Stat1 (Tyr701) Abs. Blots were stripped and reprobed with anti-Stat1 Abs to assure equal loading. (c) Induction of ISGs in UBP43+/+ and UBP43−/− bone marrow cells. Northern blot analysis of ISG15, 2′-5′OAS and IRF7 gene expression was performed on total RNA isolated from UBP43+/+ and UBP43−/− bone marrow cells treated with 100 unit/ml of IFNβ for the time indicated.

FIG. 5 shows that protein ISGylation increases IFN signaling. (a) functional UBE1L expression restores protein ISGylation in K562 cells. Control or UBE1L expression plasmid pcDNA-HA-UBE1L transiently transfected K562 cells were cultured in the absence or in the presence of 1000 units/ml IFNα for 24 hours. Protein extracts were prepared from these cells and used in the western blot analysis with antibodies against ISG15 (top panel) and HA tag (bottom panel). Clear UBE1L expression can be detected in transfected cells. * marked several non-specific signal bands. Positions of molecular weight markers are shown on the left side. (b) K562 cells were transiently transfected with p3K-UBP43-luc and either vector pcDNA or the UBE1L expression construct pcDNA-HA-UBE1L with Renilla luciferase expression construct, pRL-null for transfection efficiency control. Cells were cultured in the presence or in the absence of 1000 units/ml IFNα for the indicated time and harvested for dualluciferase assay. The promoter activity is presented as relative firefly luciferase activity, which has been normalized to Renilla luciferase activity. The average promoter activity was generated from four separate experiments. The standard deviation of the mean is indicated by the error bars.

### DEFINITIONS

An “acceptor molecule” refers to a molecule that is excited by light emitted by a donor molecule. Such molecules are known and are commercially available (Molecular Probes, Inc., Eugene Oreg., 97402).

A “donor molecule” refers to a molecule that is excited by fluorescent light and which emits light that causes excitation of an acceptor molecule. Such molecules are known and are commercially available (Molecular Probes, Inc., Eugene Oreg., 97402).

“Conservative amino acid exchange” refers to the exchange for one amino acid for another in a polypeptide chain. Preferred exchanges include, for example; aspartic-glutamic as acidic amino acids; lysine/argininelhistidine as basic amino acids; leucine/isoleucine, methionine/valine, alanine/valine as hydrophobic amino acids; serine/glycine/alanine/threonine as hydrophilic amino acids. Conservative amino acid exchange also includes groupings based on side chains. Members in each group can be exchanged with another. For example, a group of amino acids having aliphatic side chains is glycine, alanine, valine, leucine, and isoleucine. These may be exchanged with one another. A group of amino acids having aliphatic-hydroxyl side chains is serine and threonine. A group of amino acids having amide-containing side chains is asparagine and glutamine. A group of amino acids having aromatic side chains is phenylalanine, tyrosine, and tryptophan. A group of amino acids having basic side chains is lysine, arginine, and histidine. A group of amino acids having sulfur-containing side chains is cysteine and methionine. For example, replacement of a leucine with an isoleucine or valine, an aspartate with a glutamate, a threonine with a serine, or a similar replacement of an amino acid with a structurally related amino acid may be accomplished to produce peptides used in the invention.

“Variant” refers to any pharmaceutically acceptable derivative, analogue, or fragment of a polypeptide (e.g., ISG15-conjugate) described herein. A variant also encompasses one or more components of a multimer, multimers comprising an individual component, multimers comprising multiples of an individual component (e.g., multimers of a reference molecule), a chemical breakdown product, and a biological breakdown product. In particular, non-limiting embodiments, an ISG15 or ISG15-conjugate may be a “variant” relative to a reference ISG15 or ISG15-conjugate by virtue of one or more alterations in amino acid sequence, including without limitation, deletion or addition of one or more amino acid residues. In another particular, non-limiting embodiment, ISG15 or ISG15-conjugate may be chemically modified without changing the amino acid sequence of the molecule by for example, linking with targeting molecules. Accordingly, chemical modification may be used to create variants of the polypeptides of the invention that have altered charge, solubility, stability, localization, and/or targeting.

The term “ISG15-conjugate” includes polypeptides and peptidomimetics that are bound within the proteolytic site of UBP43. ISG15-conjugates may be bound and cleaved by UBP43, or may be bound but not cleaved by UBP43. An ISG15-conjugate typically includes an ISG15 portion, and a coupled partner portion. Coupled partners include, but are not limited to, phospholipase Cγ1, Jak1, ERK1, and Stat1. Accordingly, ISG15-conjugates include ISG15-phospholipase Cγ1, ISG15-Jak1, ISG15-ERK1, and ISG15-Stat1. ISG15-conjugates can also include fragments of: ISG15, a coupled partner, ISG15-phospholipase Cγ1, ISG15-Jak1, ISG15-ERK1, and ISG15-Stat1 that are bound within the proteolytic site of UBP43. In addition, ISG15-conjugates include polypeptide segments that are bound within the proteolytic site of UBP43 and cleaved. These ISG15-conjugates may include bonds that allow them to be bound by UBP43, but not proteolytically cleaved by UBP43.

The term “peptidomimetic” or “peptide mimetic” describes a peptide analog, such as those commonly used in the pharmaceutical industry as non-peptide drugs, with properties analogous to those of the template peptide. (Fauchere, J., Adv. Drug Res., 15: 29 (1986) and Evans et al., J. Med. Chem., 30: 1229 (1987)). Generally, peptidomimetics are structurally similar to a paradigm polypeptide (i.e., a polypeptide that has a biochemical property or pharmacological activity), but have one or more peptide linkages optionally replaced by a linkage such as, —CH2NH—, —CH2S—, —CH2—CH2—, —CH═CH— (cis and trans), —COCH2—, —CH(OH)CH2—, and —CH2SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data, 1: 3 (1983); Morley, J. S., Trends. Pharm. Sci., pp. 463-468 (1980); Hudson, D. et al., Int. J. Pept. Prot. Res., 14: 177-185 (1979); Spatola et al., Life Sci., 38: 1243 (1986); Harm, J. Chem. Soc. Perkini Trans I, 307-314 (1982); Almquist et al., J. Med. Chem., 23: 1392 (1980); Jennings-White et al., Tetrahedron Lett., 23: 2533 (1982); Szelke et al., European Appln. EP 45665 (1982) CA: 97: 39405 (1982); Holladay et al., Tetrahedron Lett., 24: 4401 (1983); and Hruby, Life Sci., 31: 189 (1982). Advantages of peptide mimetics over natural polypeptide embodiments may include more economical production, greater chemical stability, altered specificity, reduced antigenicity, and enhanced pharmacological properties such as half-life, absorption, potency and efficacy. Substitution of one or more amino acids within polypeptide or peptide mimetic with a D-amino acid of the same type (e.g., D-lysine in place of L-lysine) may be used to generate polypeptides and peptide mimetics that are more stable and more resistant to endogenous proteases.

A “pharmaceutical composition” includes an ISG15-conjugate or agent identified according to the invention, for example ISG15-Stat1, in combination with a pharmaceutically acceptable carrier. The ISG15-conjugates or agents of the invention may be formulated for oral or parenteral administration (e.g., by injection, for example, bolus injection or continuous infusion) and may be presented in unit dosage form in ampules, prefilled syringes, small volume infusion containers or multi-dose containers with an added preservative. The ISG15-conjugates or agents of the invention may take such forms as liposomes and may contain formulatory agents such as suspending, stabilizing and/or dispersing agents.

For topical administration to the epidermis, the ISG15-conjugates or agents of the invention may be formulated as ointments, creams or lotions, or as the active ingredient of a transdermal patch. Suitable transdermal delivery systems have been disclosed (U.S. Pat. Nos. 4,788,603; 4,931,279; 4,668,506; and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The compounds can also be delivered via iontophoresis (U.S. Pat. Nos. 4,140,122; 4,383,529; and 4,051,842).

Pharmaceutical compositions suitable for topical administration in the mouth include unit dosage forms such as lozenges comprising one or more ISG15-conjugates or agents of the invention in a flavored base, usually sucrose and acadia or tragacanth; pastilles comprising the ISG15-conjugates or agents of the invention in an inert base such as gelatin and glycerin or sucrose and acacia; mucoadherent gels, and mouthwashes comprising the ISG15-conjugates or agents of the invention in a suitable liquid carrier.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the ISG15-conjugates or agents of the invention with the softened or melted carriers) followed by chilling and shaping in molds. Typically the conjugates and agents are contained within liposomes.

Pharmaceutical compositions suitable for vaginal administration may be presented as pessaries, tampons, creams, gels, pastes, foams or sprays containing the liposome containing ISG15-conjugates or agents, and such carriers are well known in the art.

For administration by inhalation, liposomes containing ISG15-conjugates or agents according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

For intra-nasal administration, liposomes containing the ISG15-conjugates or agents of the invention may be administered via a liquid spray, such as via a plastic bottle atomizer. Typical of these are the Mistometer® (isoproterenol inhaler—Wintrop) and the Medihaler® (isoproterenol inhaler—Riker).

For topical administration to the eye, liposomes containing the ISG15-conjugates or agents can be administered as drops, gels (U.S. Pat. No. 4,255,415), gums (U.S. Pat. No. 4,136,177) or via a prolonged-release ocular insert (U.S. Pat. Nos. 3,867,519 and 3,870,791).

The amount of the ISG15-conjugates, agents, or combinations thereof that are administered and the frequency of administration to a given human patient will depend upon a variety of variables related to the patient's psychological profile and physical condition. For evaluations of these factors, see J. F. Brien et al., Eur. J. Clin. Pharmacol., 14, 133 (1978); and Physicians' Desk Reference, Charles E. Baker, Jr., Pub., Medical Economics Co., Oradell, N.J. (41st ed., 1987).

“Pharmaceutically acceptable salts” of the ISG15-conjugates or agents of the invention include, but are not limited to, the nontoxic addition salts with organic and inorganic acids, such as the citrates, bicarbonates, malonates, tartrates, gluconates, hydrochlorides, sulfates, phosphates, and the like.

“Polypeptides” and “Proteins” are used interchangeably herein. Polypeptides and proteins can be expressed in vivo through use of prokaryotic or eukaryotic expression systems. Many such expressions systems are known in the art and are commercially available. (Clontech, Palo Alto, Calif.; Stratagene, La Jolla, Calif.). Examples of such systems include, but are not limited to, the T7-expression system in prokaryotes and the bacculovirus expression system in eukaryotes. Polypeptides can also be synthesized in vitro, e.g., by the solid phase peptide synthetic method or by in vitro transcription/translation systems. The synthesis products may be fusion polypeptides, i.e., the polypeptide comprises the polypeptide variant or derivative according to the invention and another peptide or polypeptide, e.g., a His, HA or EE tag. Such methods are described, for example, in U.S. Pat. Nos. 5,595,887; 5,116,750; 5,168,049 and 5,053,133; Olson et al., Peptides, 9, 301, 307 (1988). The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). These polypeptides can be further purified by fractionation on immunoaffinity or ion-exchange columns; ethanol precipitation; reverse phase HPLC; chromatography on silica or on an ion-exchange resin such as DEAE; chromatofocusing; SDS-P AGE; ammonium sulfate precipitation; gel filtration using, for example, Sephadex G-75; or ligand affinity chromatography.

The polypeptides include the exchange of at least one amino acid residue in the polypeptide for another amino acid residue, including exchanges that utilize the D rather than L form, as well as other well known amino acid analogs, e.g., N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, omithine, citruline, N-methyl-alanine, para-benzoyl-phenylalanine, phenyl glycine, propargylglycine, sarcosine, N-acetylserine, N-formylmethionine, 3-methylhistidine, 5-hydroxylysine, and other similar amino acids and imino acids and tert-butylglycine.

The term “UBP43” refers to a polypeptide that exhibits ISG15-specific proteolytic activity. This activity is able to cleave ISG15-conjugates. The amino acid sequence of a human UBP43 has accession number NP_{—}059110, and the nucleic acid sequence has accession GeneBank accession number NM_{—}017414. The amino acid sequence of a mouse UBP43 has accession number NP_{—}036039, and the nucleic acid sequence has accession GeneBank accession number NM_{—}011909. Thus, polypeptides having amino acid deletions, additions, conservative exchanges, or combinations thereof are included within the definition of UBP43 as long as they retain the ISG15-conjugate cleavage activity exhibited by UBP43 polypeptides described by accession numbers NP_{—}059110 or NP_{—}036039.

A “substrate” refers to a polypeptide or peptidomimetic that is bound within the proteolytic site of UBP43 and which produces a detectable product when cleaved by UBP43. Examples of substrates include, but are not limited to, ISG15-phospholipase Cγ1, ISG15-Jak1, ISG15-ERK1, and ISG15-Stat1 in which the ISG15 portion of the conjugate is labeled with a first fluorescent dye and the coupled partner portion (for example, phospholipase Cγ1, Jak1, ERK1, or Stat1) is labeled with a second fluorescent dye that can undergo fluorescence resonance energy transfer (FRET) with the first fluorescent dye. Thus, cleavage of the substrate separates the first and second fluorescent dyes and causes a detectable decrease in FRET. Other examples of labels and techniques that may be used to prepare substrates and detect substrate cleavage include, but are not limited to, fluorescent quenching, spin coupling, enzyme-linked immunosorbant assay (ELISA), radio-immunoassay (RIA), and the like.

### DETAILED DESCRIPTION OF THE INVENTION

Interferons (IFNs) are important modulators of immune, inflammatory, and antiviral functions, as well as cell survival and proliferation. They exert the signals through the activation of JAK-STAT pathway that mediates rapid induction of IFN-stimulated genes (ISGs). ISG15 (or UCRP) encodes a 15 kDa protein which is strongly induced after IFN treatment and has significant sequence homology to ubiquitin. ISG15 can be conjugated to intracellular proteins via an isopeptidase bond in a manner similar to ubiquitin and other ubiquitin-like modifiers (ubls) SUMO and Nedd8.

Little was known until now about the function of protein modification by ISG15 (ISGylation) as its role in IFN signaling had not been explored. The present invention is based, in part, on the first correlation of IFN signaling and ISGylation of proteins, which is described herein. In addition, the first description of the relationship of IFN signaling, UBP43 activity, and induction of apoptosis (cellular suicide) is contained herein.

A method to determine if an agent stimulates or inhibits the expression or activity of UBP43. The present invention provides methods to identify agents that stimulate or alter the expression, level, or activity of UBP43. In particular, non-limiting embodiments, the agents increase or decrease the expression of UBP43, or activate or inhibit the protease activity of UBP43 that cleaves ISG15-conjugates.

Accordingly, in a specific nonlimiting embodiment, the invention provides a method to identify an agent that alters UBP43 activity comprising contacting a test cell with a candidate agent, determining the amount of an ISG-conjugate in the test cell, determining the amount of the ISG-conjugate in a control cell (in which the control cell is not contacted with the candidate agent), and comparing the amount of ISG15-conjugate in the test cell to the amount of ISG15-conjugate in the control cell, wherein a difference in the amount of ISG15-conjugate indicates that the agent alters UBP43 activity. The test cell, control cell, or both the test cell and control cell may express an interferon receptor. Additionally, the method may also comprise contacting the test cell, the control cell, or both the test cell and the control cell with IFNα, IFNβ, double-stranded ribonucleic acid, and/or lipopolysaccharide. The test cell and/or control cell may be an UBP43+/+ cell, UBP43± cell, or UBP43−/− cell. The ISG15-conjugate may comprise an ISG15-phospholipase Cγ1, ISG15-Jak1, ISG15-ERK1, ISG15-ERK2, ISG15-Stat1, a variant of any of these ISG15-conjugates, or any combination thereof.

In another specific nonlimiting embodiment, the invention provides a method to identify an agent that alters UBP43 activity comprising contacting a composition comprising UBP43, a substrate (for example, an ISG15-conjugate), with a candidate agent, wherein the substrate comprises a donor molecule and an acceptor molecule, determining the fluorescence resonance energy transfer between the donor molecule and the acceptor molecule; and determining the fluorescence resonance energy transfer between the donor molecule and the acceptor molecule in the composition comprising UBP43 and the substrate, without the candidate agent, wherein a difference in the fluorescence resonance energy transfer between the donor molecule and the acceptor molecule indicates that the agent alters UBP43 activity. The ISG15-conjugate, if used as the substrate, may comprise an ISG15-phospholipase Cγ1, ISG15-Jak1, ISG15-ERK1, ISG15-ERK2, ISG15-Stat1, a variant of any of these ISG15-conjugates, or any combination thereof.

In another specific nonlimiting embodiment, the invention provides a method to identify an agent that inhibits UBP43 activity comprising contacting a composition comprising UBP43, a substrate (for example, an ISG15-conjugate), with a candidate agent, wherein the substrate comprises a donor molecule and an acceptor molecule, determining the fluorescence resonance energy transfer between the donor molecule and the acceptor molecule, and determining the fluorescence resonance energy transfer between the donor molecule and the acceptor molecule in the composition comprising UBP43 and the substrate, without the candidate agent, wherein substantially no change in the fluorescence resonance energy transfer between the donor molecule and the acceptor molecule indicates that the substrate is not being cleaved by UBP43, and thus indicating that UBP43 activity is inhibited. The ISG15-conjugate, if used as the substrate, may comprise an ISG15-phospholipase Cγ1, ISG15-Jak1, ISG15-ERK1, ISG15-ERK2, ISG15-Stat1, a variant of any of these ISG15-conjugates, or any combination thereof.

In one embodiment, an agent that decreases expression or the level of UBP43, or inhibits the protease activity of UBP43, increases the concentration of an ISG15-conjugate within a cell. The increased concentration of the ISG15-conjugate within the cell causes the cell to undergo apoptosis. Methods to determine apoptosis are known in the art and include terminal deoxyribosyl-transferase mediated dUTP nick end-labeling (TUNEL). In a particular embodiment, the agent causes the cell to become hypersensitive to interferon.

Accordingly, in a specific non-limiting embodiment, the present invention provides a method to induce a cell to undergo apoptosis, or accelerate apoptosis in a cell, comprising contacting a cell with a composition comprising an ISG15-conjugate. The ISG15-conjugate may comprise an ISG15-phospholipase Cγ1, ISG15-Jak1, ISG15-ERK1, ISG15-ERK2, ISG15-Stat1, a variant of any of these ISG15-conjugates, or any combination thereof. Additionally, the method may also comprise contacting the test cell, the control cell, or both the test cell and the control cell with IFNα, IFNβ, double-stranded ribonucleic acid, and/or lipopolysaccharide. In particular embodiments, the ISG15-conjugate cannot be cleaved by UBP43, and/or the ISG15-conjugate includes a non-hydrolysable bond. The composition may further comprises a pharmaceutically acceptable carrier, for example, within a liposome. Thus, in a specific nonlimiting embodiment, the invention provides a method to promote or increase apoptosis in a cell comprising contacting the cell with a composition comprising an agent that inhibits UBP43 activity. The presence or amount of cellular apoptosis can be measured to determine whether apoptosis has been promoted or increased.

In another embodiment, an agent that increases expression or the level of UBP43, or increases the protease activity of UBP43, decreases the concentration of an ISG15-conjugate within a cell. The decreased concentration of the ISG15-conjugate within the cell inhibits differentiation of the cell and/or causes the cell to undergo multiple rounds of replication. In a particular embodiment, the agent causes the cell to become hyposensitive to interferon.

The concentration of ISG15-conjugates within a cell can be determined through use of numerous immunological assays. For example, western blots using antibodies against ISG15, or the coupled partner, maybe used to detect ISG15-conjugates (Padovan et al., Cancer Res., 62: 3453 (2002); Malakhova et al., J. Biol. Chem., 277: 14703 (2002); D'Cunha et al., J. Immunol., 157: 4100 (1996)). Western blots can also be combined with immunoprecipitation methods to determine the concentration of ISG15-conjugates within a cell.

UBP43 messenger RNA production (mRNA) within a cell may be determined through use of numerous methods, such as northern blots, RNase protection assays, fluorescent hybridization, and the like (Kanagawa, AIST Today, 2: 32 (2002); Torimura et al., Analytical Sciences, 17: 155 (2001); Malakhova et al., J. Biol. Chem., 277: 14703 (2002). UBP43 protein production within a cell may be determined through use of numerous immunological methods, such as western blots, ELISA assays, radioimmunoassays, and the like (Sambrook et al., Molecular Cloning: A Laboratory Manual, 3rd edition, Cold Spring Harbor Press, Cold Spring Harbor, N.Y. (2001); Harlow et al., Antibodies: A Laboratory Manual (Cold Spring Harbor Pub. 1988)).

Additionally, cells have been created that are UBP43^{-/-} and therefore do not produce UBP43 mRNA or protein. These cells can be maintained in culture in the absence of interferon stimulation even though they do not express UBP43. UBP43^{-/-} cells may be used in accordance with the methods of the invention to determine if a candidate agent regulates upstream or downstream factors that regulate the production of UBP43 mRNA or protein.

Cell-based Assays. In one example, the method provides a cell-based method to determine if a candidate agent alters the activity of UBP43. In a particular embodiment, the method comprises contacting a UBP43^{+/+} test cell with a candidate agent and then determining if the concentration of an ISG-conjugate within the test cell is altered (e.g., increased or decreased) when compared to the ISG15-conjugate concentration in a UBP43^{+/+} control cell that was not contacted with the candidate agent. As an additional control, a UBP43^{-/-} cell can be contacted with the candidate agent and the concentration of an ISG15-conjugate within the contacted UBP43^{-/-} cell can be compared to the conjugate concentration in a UBP43^{-/-} cell that was not contacted with the candidate agent. In particular, non-limiting embodiments, an increase or decrease in the concentration of ISG15-conjugate in a contacted UBP43^{+/+} test cell versus non-contacted UBP43^{+/+} control cell indicates that the agent stimulates or inhibits, respectively, the expression, level, or activity of UBP43. The specificity of action of the candidate agent toward UBP43 can be confirmed by examination of the results of a similar assay conducted in UBP43^{-/-} cells. For example, if a candidate agent activates or inhibits a factor (such as JAK1, Tyk2, STAT1, STAT2, p48, or IRF9) that is an upstream activator of ISG15, and conjugates thereof, the ISG15-conjugate concentration will be increased or decreased within the UBP43^{-/-} cell contacted with the candidate agent when compared to the ISG15-conjugate concentration within the UBP43^{-/-} cell that was not contacted with the candidate agent. However, if the ISG15-conjugate concentration in the UBP43^{-/-} cell that was contacted with the candidate agent is the same as the ISG15-conjugate concentration in the UBP43^{-/-} cell that was not contacted with the candidate agent, it demonstrates that the candidate agent does not affect upstream activators or inhibitors of the production of the ISG15-conjugate. Accordingly, if the candidate agent does not alter ISG15-conjugate concentration in the UBP43^{-/-} cells, but alters ISG15-conjugate concentration in the UBP43^{+/+} cells, the candidate agent specifically acts on UBP43. The specificity of the action of the candidate agent on UBP43 can then be further confirmed through use of in vitro assays as described herein.

In vitro assays. In another example, the invention provides in vitro methods to determine if a candidate agent alters (e.g., activates or inhibits) the ability of UBP43 to cleave an ISG15-conjugate. In particular embodiments, the in vitro methods comprise incubating UBP43 with an ISG15-conjugate, in the presence and/or absence of a candidate agent, under conditions wherein the UBP43 can cleave the ISG15-conjugate. Cleavage of the ISG15-conjugate can be determined through use of a variety of methods (Malakhov et al., J. Biol. Chem., 277: 9976 (2002)). For example, western blotting may be used to detect cleavage of the ISG15-conjugate with antibodies that recognize ISG15, or the coupled partner. Conditions wherein UBP43 is active in vitro are known and have been described (Malakhov et al., J. Biol. Chem., 277: 9976 (2002)).

The present invention also provides a high-throughput assay to identify activators or inhibitors of UBP43-mediated substrate cleavage. In particular, non-limiting embodiments, the methods of using the assay comprise detecting cleavage of a substrate by UBP43 through detection of a signal that is altered (e.g., increased or decreased) upon cleavage of the UBP43 substrate. In particular, non-limiting embodiments substrates are used that have attached thereon two label molecules that are separated by a site that is proteolytically cleaved by UBP43, or a variant thereof (e.g.; an ortholog), such that a detectable signal is produced upon separation of the two label molecules by cleavage of the substrate at the cleavage site. Examples of such label molecules include, but are not limited to, molecules used for fluorescence resonance energy transfer (FRET), fluorescent quenching, spin labels, and the like.

For example, fluorescence resonance energy transfer (FRET) may be used to detect cleavage of a substrate by UBP43. Fluorescence energy transfer is a distance-dependent interaction between the electronic excited states of two dye molecules in which excitation is transferred from a donor molecule to an acceptor molecule. The efficiency of FRET is dependent on the inverse sixth power of the intermolecular separation, making it useful over distances comparable with the dimensions of biological macromolecules. Typically, a donor and an acceptor molecule are within from 10-100 angstroms of each other. Typical examples of donor and acceptor molecule pairs include: fluorescein-tetramethylrhodamine, IAEDANS-fluorescein, EDANS-Dabcyl, fluorescein-fluorrescein, BODIPY FL-BODIPY FL, fluroescein-QSY 7 dye, and fluroescein-QSY 9 dye. Dyes and instructions for their use are commercially available and known in the art (Molecular Probes, Inc., Eugene Oreg,. 97402). Accordingly, a substrate can be prepared having a donor molecule and an acceptor molecule attached such that cleavage by UBP43 will separate the donor molecule from the acceptor molecule. This separation will cause a detectable decrease in FRET that can be monitored to determine the proteolytic activity of UBP43 on a substrate. An example of a suitable substrate is any substrate that can be labeled with a molecule useful for FRET and one which is cleaved by UBP43, such as a dye labeled ISG15-conjugate. UBP43 substrates have been described and can be readily labeled for use with FRET through use of methods well known in the art (Sambrook et al., Molecular Cloning: A Laboratory Manual, 3rd edition, Cold Spring Harbor Press, Cold Spring Harbor, N.Y. (2001); Malakhov et al., J. Biol. Chem., 277: 9976 (2002)). These methods can be used to determine if a candidate agent inhibits the proteolytic activity of UBP43. These methods may be adapted for use in automated systems for high-throughput screening of candidate agents. For example, a fluorimetric imaging plate readers (FLIPR) may be used. These automated methods are known in the pharmaceutical industry. For example, robotic arms may be used to transfer a FRET substrate, a candidate agent, and UBP43 into a well of a 96 well plate. Following incubation, fluorescence resonance energy transfer within the wells of the plate may be detected through use of a plate reader to identify candidate agents that decrease the activity of UBP43. Numerous automated systems may be constructed to measure the activity of UBP43 through use of fluorescence resonance energy transfer based methods.

Fluorescent quenching may also be used to determine whether a candidate agent is an activator or inhibitor of UBP43 proteolytic activity. Generally, these methods use a UBP43 substrate having attached thereon a fluorescent molecule and a fluorescent quenching molecule. The fluorescent molecule and the fluorescent quenching molecule are linked to the substrate in close enough proximity to each other that the fluorescence of the fluorescent molecule is quenched. However, upon separation of the fluorescent molecule and the fluorescent quenching molecule by cleavage of the substrate, the fluorescence of the fluorescent molecule is no longer quenched and an increase in fluorescence can be detected. Accordingly, the ability of a candidate agent to decrease the proteolytic activity of UBP43 can be determined, for example, by detecting whether the presence of the candidate agent causes a decrease in fluorescence by inhibiting cleavage of the substrate. These methods may be readily adapted for use within automated systems. For example, a robotic arm can be used to dispense a labeled substrate, and UBP43 into a control well of a 96 well plate; and a labeled substrate, UBP43, and candidate agents into the remaining test wells of the 96 well plate. The plate can be incubated and then a fluorescent plate reader can be used to determine the increase in fluorescence in the control well and any increase in fluorescence in the test wells. The level of fluorescence in a test well can be compared to fluorescence in the control well to determine if the candidate agent added to the test well inhibited the proteolytic activity of UBP43 as indicated by decreased fluorescence. Numerous fluorescent molecules and quenchers are known in the art and are commercially available (Epoch Biosciences, Bothell, Wash., 98021; Aldrich, Milwaukee, Wis., 53201).

Method to induce cells to undergo apoptosis and to treat diseases related to cellular proliferation. The present invention provides methods to induce a cell to undergo apoptosis and/or to enhance apoptosis in a cell. These methods generally relate to the discovery disclosed herein that inhibiting the activity of UBP43 causes cell apoptosis. In particular, non-limiting embodiments, these methods increase the ISG15-conjugate concentration within a cell induces the cell to undergo apoptosis. The ISG15-conjugate concentration may be increased within a cell by introducing an ISG15-conjugate into the cell. In other particular, non-limiting embodiments, these methods cause the cell to become hypersensitive to interferon. The ISG15-conjugate concentration may also be increased within a cell by inhibiting the UBP43 enzyme that degrades the ISG15-conjugate. UBP43 hydrolyzes the bond that links ISG15 and the target protein. Alternatively, increasing UBE1L activity can increase the ISG15-conjugate concentration within the cell by causing more ISG15 to be linked to a conjugate. This may be done, for example, by treating cells with all-trans-retinoic acid which may induce UBE1L (Ketareewan et al., Proc. Natl. Acad. Sci., 99: 3806 (2002)). Accordingly, described herein below are methods and formulations for directly introducing ISG15-conjugates into a cell, and methods for inhibiting the activity of UBP43 to induce a cell to undergo apoptoses. These methods may also be combined with the administration of retinoic acid, which may induce UBE1L and cause a cell to undergo apoptosis.

ISG15-conjugates. ISG15 is a ubiquitin-like protein that conjugates to numerous proteins in cells. Examples of such ISG15-conjugates include ISG15-phospholipase Cγ1, ISG15-Jak1, ISG15-ERK1, and ISG15-Stat1, as described in U.S. Application No. 60/444,941, the which is herein incorporated by reference in its entirety. These conjugates can be prepared through use of recombinant techniques and purified from cells. Alternatively, these conjugates can be prepared by incubation of ISG15 with the coupled partner of the conjugate and UBE1L under conditions wherein the coupled partner will be linked to ISG15.

Any single, or combination of, ISG15-conjugates can be administered to a cell to induce the cell to undergo apoptosis. Apoptosis if the cell is thought to be a result of the cell becoming hypersensitive to interferon stimulation. The ISG15-conjugates may also be administered to a cell in combination with other agents and compounds. For example, the ISG15-conjugates can be administered in combination with interferon, double stranded ribonucleic acid, or lipopolysaccharide. The ISG15-conjugates may also be administered in combination with other therapeutic agents. Examples of such therapeutic agent may be found in numerous formularies (for example: Merck Index, Physicians' Desk Reference, 55 ed., (2001), Medical Economics Company, Inc., Montvale, N.J.; USPN Dictionary of USAN and International Drug Names, (2000), The United States Pharmacopeial Convention, Inc., Rockville, Md.; and The Merck Index, 12 ed., (1996), Merck & Co., Inc., Whitehouse Station, N.J.). Furthermore, the ISG15-conjugates may be administered in combination with UBP43 inhibitors that are identified according to the methods described herein.

ISG15-conjugates may be prepared synthetically and can therefore contain chemical linkages other than peptide bonds. Thus, the ISG15-conjugates can be peptidomimetics. An advantage of such conjugates is that they are often resistant to degradation by proteases and therefore have a longer half-life.

ISG15-conjugate based UBP43 inhibitors. The invention provides methods to identify UBP43 inhibitors. These inhibitors can be derived from fragments of ISG15-conjugates that are bound by UBP43. The fragments may be determined by proteolytic digestion of ISG15-conjugates, followed by purification of the produced fragments. Numerous methods may be used to purify proteolytic fragments, such as high-pressure liquid chromatography (HPLC), fast protein liquid chromatography (FPLC), gel chromatography, and the like. The fragments can then be screened using the methods disclosed herein to determine fragments that are able to inhibit UBP43 activity. Fragments able to inhibit the proteolytic activity of UBP43 can be characterized to determine their composition through use of common techniques. Such techniques include additional proteolytic characterization, electrospray mass-spectroscopy, susceptibility to chemical degradation or derivatization, and the like. Once the compositions of the inhibitory fragments are determined, they can be synthesized. Analogs of the fragments can also be synthesized that contain bonds other than peptide bonds. Alternatively, existing chemical compounds can be screened for their ability to inhibit the activity of UBP43. Rational drug design can also be used to design compounds that are able to inhibit the activity of UBP43 based on crystal structure analysis.

Formulations and administration of an ISG15-conjugate, agent, or ISG15-conjugate based UBP43 inhibitor to a cell. One or more ISG15-conjugates may be introduced into cells through use of liposomes. This method involves encapsulating the ISG15-conjugate within a liposome. The liposome can then be contacted with a cell such that the liposome fuses with the cellular membrane and releases the encapsulated ISG15-conjugate into the cell. Methods of preparing liposomes for delivering therapeutic agents to cells are known and have been described (U.S. Pat. Nos. 4,356,167; 4,873,088; 6,290,987; 6,221,382). The liposomes containing one or more ISG15-conjugates can be administered to a cell through a variety of routes that include injection; direct application to a surface, such as skin or a mucosal surface; aerosols for application to lung tissue, and the like. The ISG-conjugates can also be administered in combination with agents identified according to the invention, and with other known therapeutic agents that are known in the art, such as those described herein.

Methods for treating disease related to cellular proliferation. The ISG15-conjugates, agents of the invention, and pharmaceutical compositions described herein can be used to induce cells to undergo apoptosis. Accordingly, the present invention provides a method to inhibit cell proliferation comprising contacting a cell with a composition comprising an ISG15-conjugate (experimental), detecting the amount of cell proliferation of the cell, and comparing the amount of cell proliferation detected in the experimental with a control amount of cell proliferation (which can be determined from the amount of proliferation of a cell not contacted with the composition), wherein a decrease in the amount of cell proliferation detected in the experimental compared to the control amount indicates that the composition inhibits cell proliferation The ISG15-conjugate may be, for example, ISG15-phospholipase Cγ1, ISG15-Jak1, ISG15-ERK1, ISG15-Stat1, and/or a variant thereof. The composition may further comprise a pharmaceutically acceptable carrier. The composition may be contained within a liposome. An interferon, double-stranded ribonucleic acid, and/or lipopolysaccharide may also be contacted with the cell. The cell may be a tumor cell.

The present invention also provides a method to induce or increase apoptosis in a cell comprising contacting a cell with a composition comprising an ISG15-conjugate (experimental), detecting the amount of cellular apoptosis, and comparing the amount of cellular apoptosis detected in the experimental with a control amount of cellular apoptosis (which can be determined from the amount of apoptosis of a cell not contacted with the composition), wherein an increase in the amount of cellular apoptosis detected in the experimental compared to the control amount indicates that the composition induces or increases apoptosis The ISG15-conjugate may be, for example, ISG15-phospholipase Cγ1, ISG15-Jak1, ISG15-ERK1, ISG15-Stat1, and/or a variant thereof. The composition may further comprise a pharmaceutically acceptable carrier. The composition may be contained within a liposome. An interferon, double-stranded ribonucleic acid, and/or lipopolysaccharide may also be contacted with the cell. The cell may be a tumor cell.

In other non-limiting embodiments, the present invention provides methods for treating diseases related to cellular proliferation. Such diseases may be caused by viral infection. Examples of such diseases and their causative virus include: cervical carcinoma (human papillomaviruses); Karposi's sarcoma (cytomegalovirus); Burkitt's lymphoma, immunoblastic lymphoma, nasopharyngeal carcinoma (Epstein-Barr virus); hepatocellular carcinoma (hepatitis B virus); and human T-cell lymphoma (human retroviruses). Many proliferative diseases are known to be due to chromosomal abnormalities. Such diseases are exemplified by myeloid leukemia, malignant lymphoma, lymphocytic leukemia, and myeloproliferative diseases (Merck Manual, Merck Research Laboratories, Whitehouse Station, N.J. (1999)). Thus, the invention includes methods wherein ISG15-conjugates, agents of the invention, and combinations thereof can be administered alone or in combination with known therapeutics, such as retinoic acid, to patients in need thereof. Such an administration scheme will cause the contacted cells to undergo apoptosis and die.

Accordingly, the invention may also be used to increase the effectiveness of anticancer therapeutics already in use, such as radiation therapy and chemotherapy. In a particular nonlimiting embodiment, the invention provides a method to increase the effectiveness of an anticancer therapeutic comprising administering to a patient in need thereof an effective amount of the anticancer therapeutic and a composition comprising an ISG15-conjugate or a composition comprising an agent that inhibits UBP43. The anticancer therapeutic can be administered before, after, concurrently, or overlapping with the administration of the composition.

In a specific nonlimiting embodiment, the invention provides a method to increase the effectiveness of an anticancer therapeutic comprising administering to a patient in need thereof an effective amount of the anticancer therapeutic, administering to the patient an effective amount of a composition comprising an ISG15-conjugate. The effectiveness of the co-administration of the anticancer therapeutic and the composition can be compared with a control degree of effectiveness (which can be determined from the effectiveness of the anticancer therapeutic when administered alone or in the absence of the ISG15-conjugate) wherein an increase in the effectiveness of the co-administration compared to the control degree of effectiveness indicates that the composition increases the effectiveness of the anticancer therapeutic. In specific nonlimiting embodiments, the ISG15-conjugate is ISG15-phospholipase Cγ1, ISG15-Jak1, ISG15-ERK1, ISG15-ERK2, ISG15-Stat1, a variant of any of these ISG15-conjugates, or any combination thereof.

Methods to prolong and increase interferon action on a cell. The invention provides methods to prolong and increase interferon action on a cell. This aspect of the invention is based on the discovery that inhibition of UBP43 activity, or other mechanisms through which the ISG15-conjugate concentration in the cell is increased, produces prolonged and increased interferon action on the cell. Accordingly, the ISG15-conjugates and agents of the invention can be administered to a cell to increase and prolong the responsiveness of the cell to interferon. Such methods have tremendous utility for treating diseases in which prolonged interferon response or the induction of apoptosis is desirable. Examples of such diseases include cancer, chronic viral infection, autoimmune diseases, and the like. Thus, an ISG15-conjugate or agent of the invention can be administered alone; in combination with an agent of the invention; in combination with a known therapeutic; or in combination with an interferon inducing agent, such as double stranded ribonucleic acid or lipopolysaccharide; to prolong and increase the effects of interferon treatment.

Accordingly, in a specific nonlimiting embodiment, the invention provides a method to prolong or increase the response of a cell to interferon comprising contacting the cell with a composition comprising an ISG15-conjugate or a composition comprising an agent that inhibits UBP43 activity. In specific nonlimiting embodiments, the ISG15-conjugate is ISG15-phospholipase Cγ1, ISG15-Jak1, ISG15-ERK1, ISG15-ERK2, ISG15-Stat1, a variant of any of these ISG15-conjugates, or any combination thereof. In particular nonlimiting embodiment, the ISG15-conjugate cannot be cleared by UBP43. The method may also comprise comprising contacting the cell with IFNα, IFNβ, double-stranded ribonucleic acid, and/or lipopolysaccharide. The composition may also comprise a pharmaceutically acceptable carrier, and for example, be contained within a liposome.

Method to treat multiple sclerosis (MS). Multiple sclerosis is an autoimmune disease that affects the central nervous system. Individuals with this disease produce autoreactive T cells that participate in the formation of inflammatory lesions along the myelin sheath of nerve fibers. The cerobrospinal fluid of patients with active multiple sclerosis contains activated T lymphocytes, which infiltrate the brain tissue and cause characteristic inflammatory lesions, destroying the myelin. Since myelin functions to insulate the nerve fibers, a breakdown in the myelin sheath leads to numerous neurologic dysfunctions (Kuby, Imunology, 4th Ed., W. H. Freeman and Company, New York (2000)). Beta interferons are currently being used to treat relapsing-remitting forms of the disease. Use of bea interferons can reduce the relapse rate by approximately 3% and also reduce the risk of disability progression. Beta interferons have also been found to favorably influence brain MS induced brain lesions as judged by MRI (Vennersch et al., Rev. Med. Interne., 23 Suppl. 4: 475 (2002); Corboy et al., Curr. Treat. Options. Neurol., 5: 35 (2003)).

The ISG15-conjugates and agents described herein can be used to treat multiple sclerosis by prolonging and increasing the effects of interferon on cells. Administration of the ISG15-conjugates, agents, and combinations thereof can be conducted as described herein. For example, liposomes containing the ISG15-conjugates, agents, and combinations thereof can be applied directly to cells through injection alone or in combination with known therapeutic agents, such as interferon. Alternatively, the ISG15-conjugates, agents, and combinations thereof can be administered to a patient in need thereof through use of other administration routes as are known or described herein.

Accordingly, in a particular nonlimiting embodiment, the method comprises administering to a patient in need of thereof an effective amount of a composition comprising an ISG15-conjugate or a composition comprising an agent that inhibits UBP43 activity. An effective amount is that which results in an ameliorative response.

Method to alter phagocytosis. The present invention provides a method to alter phagocytic activity of a cell. In a particular nonlimiting embodiment, the method comprises contacting a cell with a composition comprising an ISG15-conjugate or a composition comprising an agent that inhibits UBP43 activity. After exposure of the cell to the composition, the amount of phagocytic activity of the cell can be measured to determine whether phagocytic activity has been altered. Accordingly, in a specific nonlimiting embodiment, the invention provides a method to increase phagocytic activity of a cell comprising contacting a cell with a composition comprising an ISG15-conjugate, comparing the amount of phagocytic activity detected in the experimental with a control amount of phagocytic activity (which can be determined from the amount of phagocytic activity of a cell not contacted with the composition), wherein an increase in the amount of phagocytic activity detected in the experimental compared to the control amount indicates that the composition increases phagocytic activity.

Method to alter cell motility. The present invention provides a method to alter the motility of a cell. In a particular nonlimiting embodiment, the method comprises contacting a cell with a composition comprising an ISG15-conjugate or a composition comprising an agent that inhibits UBP43 activity. After exposure of the cell to the composition, the amount of cell motility can be measured to determine whether cell motility has been altered. Accordingly, in a specific nonlimiting embodiment, the invention provides a method to increase cell motility comprising contacting a cell with a composition comprising an ISG15-conjugate, comparing the amount of cell motility detected in the experimental with a control amount of cell motility (which can be determined from the amount of cell motility not contacted with the composition), wherein an increase in the amount of cell motility detected in the experimental compared to the control amount indicates that the composition increases cell motility.

Method to promote or enhance wound healing. The present invention provides a method to accelerate wound healing. In a particular nonlimiting embodiment, the method comprises contacting a cell with a composition comprising an ISG15-conjugate or a composition comprising an agent that inhibits UBP43 activity. After exposure of the cell to the composition, the rate of wound healing can be measured to determine whether wound healing has been accelerated or otherwise enhanced. Accordingly, in a specific nonlimiting embodiment, the invention provides a method to promote or enhance wound healing comprising contacting a cell with a composition comprising an ISG15-conjugate, comparing the rate of wound healing detected in the experimental with a control rate of wound healing. The control rate can be determined from the amount of cell motility not contacted with the composition. An increase in the rate of wound healing detected in the experimental compared to the control amount indicates that the composition promotes or enhances wound healing.

Method to inhibit viral activity. The present invention provides a method to inhibit viral activity. For example, a composition of the invention may be used to inhibit the infection of a cell by a virus, or inhibit replication of a virus in a cell. Accordingly, in a particular nonlimiting embodiment, the method comprises contacting a cell with a composition comprising an ISG15-conjugate or a composition comprising an agent that inhibits UBP43 activity. After exposure of the cell to the composition, the amount of viral infection or viral replication, for example, can be measured to determine whether viral infectivity or replication has been inhibited. Accordingly, in a specific nonlimiting embodiment, the invention provides a method to inhibit viral infection or viral replication comprising contacting a cell with a composition comprising an ISG15-conjugate. The amount of resulting viral infection or viral replication can be compared to the control amount of viral infection or viral replication (i.e., in the absence of the composition), wherein a decrease in the amount of viral infection or viral replication that is detected in the experimental compared to the control amount indicates that the composition has an inhibitory effect on viral infection and/or viral replication.

In the above embodiments of the present invention, an agent that inhibits UBP activity may do so directly (e.g., by irreversible binding) or indirectly, for example, by decreasing UBP43 expression, decreasing stability of the UBP43 protein, or promoting degradation of UBP43.

### EXAMPLES

The protease that specifically cleaves ISG15, UBP43 (also known as USP18 or ISG43), belongs to the ubiquitin-specific protease (UBP or USP) family. The expression of UBP43 is strongly activated by IFN or LPS treatment. Thus, UBP43 knockout mice were generated to explore the function of protein ISGylation.

The UBP43−/− mice were viable at birth but gradually manifested neurological disorders associated with the development of hydrocephalus. Despite the relevance of UBP43 to the development of hematopoietic system, analysis of peripheral and bone marrow blood cells of UBP43-null mice did not reveal any significant defect. Both UBP43 expression and conjugation of ISG15 were strongly induced by IFN, indicating that the level of ISG15-conjugates is tightly controlled. To determine if ISGylation may affect cellular response to IFN, the phenotype of UBP43 knockout mice in response to IFN stimulation was determined. The UBP43 deficient cells were sensitive to type I IFN and underwent apoptosis upon IFN stimulation. Lack of UBP43 activity resulted in an increase in the level of protein ISGylation that is associated with the enhanced and prolonged JAK-STAT signaling. Furthermore, the role of ISGylation in positive regulation of IFN signaling was also confirmed in reconstitution assays of UBE1L-deficient K562 cells. Thus, these results indicate that UBP43 may act as a negative feedback in the IFN activated signaling pathway by decreasing levels of protein ISGylation.

DNA Constructs: pcDNA6-UBP43 construct for expression UBP43-V5-6His fusion in mammalian cells has been previously described (Schwer et al., Genomics, 65: 44 (2000)). Plasmids expressing the ubiquitin-specific proteases UBP41 (as GST fusion), UBPI (Tobias and Varshavsky, J. Biol. Chem., 266: 12021 (1991)), Unp, and Unp(mut) (Gilchrist and Baker, Biochem. Biophys. Acta., 1481: 297 (1994)) as well as pUb-GSTP1 (Baker et al., J. Biol. Chem., 269: 25381 (1994)) construct were provided by Dr. R. Baker (Australian National University, Canberra). pGEX-Nedd8-gsPESTc and pGEX-SUMO-gsPESTc plasmids were from Dr. K. Tanaka (The Tokyo Metropolitan Institute of Medical Science, Japan) (Kawakami et al., J. Biochem. (Tokyo), 126: 612 (1999)). pGEX-ISG15-Rcap and pRSVISG17 plasmids were received from Dr. A. L. Haas (Medical College of Wisconsin, Milwaukee, Wis.). To produce pGEX-ISG15-gsPESTc and pGEX-ISG17-gsPESTc expressing GST-ISG15-gsPESTc or GST-pro-ISG15-gsPESTc, respectively, the plasmid pGEX-Nedd8-gsPESTc was digested with Bamill and the excised Nedd8 was replaced with ISG15 or ISG 17 that had been PCR amplified from pGEX-ISG15-Rcap or pRSV-ISG17. To produce pET-Ub-gsPESTc plasmid expressing Ub-gsPESTc fusion the gsPESTc-coding sequence was excised from pGEX-Nedd8-gsPESTc using BamBI and EcoRI and cloned into pET-Ub-UBP43-H plasmid (see below) from which UBP43-H encoding sequence had been excised.

Construction of pGEX-UBP43 (pGEX-4T-3-UBP43) has been previously described (Liu et al., Mol. Cell. Biol., 12: 3029(1999)). pET-S-UBP43-H was produced by cloning of the full-length UBP43 gene into pET29a vector (Novagen, Madison, Wis.). To generate a construct expressing GST-UBP43-6His the pET-S-UBP43-H was digested with SacI and Smal restriction endonucleases to excise part of UBP43 together with the fused His6 tag. The resulting SacI-SmaI fragment was cloned into pGEX-UBP43 (mut) digested with SacI and NotI (blunted). pET-Ub-UBP43-H and pET-ISG15-UBP43-H constructs expressing the fusions of Ub or ISG15 with UBP43 were produced by replacing the S-tag part in pET-S-UBP43-H (digested with NdeI and EcoR V) with Ub or ISG15 genes that had been PCR amplified from pUb-GSTP1 or pGEX-ISG15-Rcap plasmids.

Inactive form of UBP43 was obtained by conversion of a critical cysteine residue (Cys⁶¹) into serine by site-directed mutagenesis of pBK/CMV-UBP43 plasmid (previously described (Liu et al., Mol. Cell. Biol., 12: 3029 (1999)) using the QuikChange kit (Stratagene, La Jolla, Calif.). To convert the constructs expressing the wild-type version of UBP43 into inactive forms, internal SacI-XbaI fragments of the UBP43 gene in the respective constructs were replaced with SacI-XbaI fragments of UBP43(mut) excised from pBK-CMV-UBP43 (mut). All constructs generated in the course of this work were sequenced.

Radioiodinated Substrates for in Vitro Enzymatic Assays. The plasmids expressing UbI-gsPESTc constructs were transformed into BL21(DE3) E. coli and the expression was induced with isopropyl-1-thio-β-D-galactopyranoside. GST-Nedd8-gsPESTc and GST-SUMO-gsPESTc fusions were purified on GSH-agarose (Sigma), digested with thrombin protease (Amersham Biosciences, Inc., Piscataway, N.J.) while attached to GSH-agarose beads and the supernatants containing these Ubls-gsPESTc were directly used for labeling. GST-ISG15-gsPESTc fusion was purified on GSH-agarose and eluted with reduced glutathion. After dialysis GST-ISG15-gsPESTc was digested with thrombin protease and the solution containing both GST and ISG15-gsPESTc polypeptides used for labeling. Ub-gsPESTc fusion was purified by heat denaturation, (NH4)2SO4 precipitation and ion-exchange chromatography as described (Kawakami et al., J. Biochem. (Tokyo), 126: 612 (1999)). The UbI-gsPESTc fusions were radiolabeled with Na¹²⁵I (ICN, Costa Mesa, Calif.) using IODO-BEADS (Pierce, Rockford, Ill.) for 5 minutes in accordance with the manufacturer‘s instructions. The labeling conditions used resulted in almost exclusive labeling of gsPESTc extension.

Puriflcation of UBP43 from E. coli. Expression of UBP43 in E. coli as either GST-UBP43 or UBP43-6His fusions produced less than 30% of full-length UBP43. Therefore the UBP43 gene was expressed as GST-UBP43-6His fusion (both wild-type and mutant versions). One liter of BL21 E. coli culture transformed with pGEX-UBP43-H plasmid was grown in LB broth to A₆₀₀=0.8 and protein expression was induced with isopropyl-1-thio-β-D-galactopyranoside. After 3 hours of induction at 21° C., cells were harvested by centrifugation, washed with PBS, and lysed by sonication in PBS adjusted to 0 mM imidazole, 300 mM NaCl, 2 mM β-mercaptoethanol, 2 mM phenylmethylsulfonyl fluoride. Lysate was cleared by 10 minutes centrifugation at 20,000×g and GST-UBP43-6His fusions were absorbed on 3 ml of Ni—NT A-agarose (Qiagen, Valencia, Calif.). After elution with PBS containing 300 mM NaCl, 250 mM imidazole, and 0.1% n-octyl β-D-glucopyranoside, the fusions were absorbed to 0.6 ml of GSH-agarose. The resin was washed with PBS, 0.1% n-octyl P-β-glucopyranoside, resuspended in 10 mM Tris, pH 7.5, 30% glycerol and stored at −20° C. until use. All purification procedures were performed at +4° C.

Purification of UBP43 from 293T Cells. 2×10⁶ of 293T cells were seeded on 60-mm cell culture plates and transfected with pcDNA6-UBP43 plasmids using Polyfect reagent (Qiagen) according to the manufacturer's instructions. Thirty-six hours after transfection cells were washed with PBS and harvested into 1 ml of lysis buffer (PBS adjusted to 10 mM imidazole, 0.1% n-octyl β-D-glucopyranoside, 300 mM NaCI, 2 mM β-mercaptoethanol, 2 mM phenyhnethylsulfonyl fluoride, and 10 μg/ml leupeptin) and then lysed by a 5-s pulse of sonication. Lysate was cleared by a 5-min centrifugation at 16,000×g and UBP43-V5-6His fusions were absorbed to 40 μl of Ni—NTA-agarose for 1 h. Beads were washed once with 1 ml of PBS adjusted to 300 mM NaCI, 20 mM imidazole, and 0.1% n-octyl β-D-glucopyranoside, twice with the same buffer containing 4 M urea, and twice with the buffer with no urea. Beads were resuspended in 80 μl of 10 rom Tris, pH 7.5,5 mM EDTA, 5 mM dithiothreitol, 20 mM imidazole, and 0.1% n-octyl β-D-glucopyranoside. The suspensions (15 μl per enzymatic reaction, 1 μg of total protein, and 0.1 μg of UBP43 fusion) were immediately used for enzymatic assay. Equal efficiency of expression, and purification between wild-type and mutant UBP43s was confirmed by Western blot with anti-V5 antibodies (Invitrogen).

Assays of UBP43 Activity in E. coli. To assess the ability of Ub-UBP43 and ISG15-UBP43 fusions to undergo self-hydrolysis, E. coli were transformed with pET-Ub-UBP43˜H and pET-ISG15-UBP43-H carrying wild-type or inactive versions of UBP43. Production of LBP43 in E. coli grown in rich media, such as LB, resulted in high yield but massive (more than 70%) degradation of UBP43. On the contrary, growth of E. coli in nutrient-poor medium M9 and induction of expression at low temperature, resulted in production of small quantities of full-length UBP43. Therefore, in this experiment, E. coli cultures were grown in 5 ml of M9 minimal medium to A₆₀₀=0.8 and induced with isopropyl-1-thio-β-D-galactopyranoside for 4 h at 21° C. Cells were harvested, lysed by sonication, and after removal of insoluble material at 18,000×g for 5 min, supernatants were resolved on 10% SDS-P AGE, electroblotted, and then probed with antibodies against the NH₂-terminal part of UBP43.

Antiproliferative and Antiviral Assays. Antiproliferative (Johns et al., J. Natl. Cancer Inst., 84: 1185 (1992)) and antiviral assays (Wines et al., Biochem. Mol. Biol. Int., 11: 1111 (1993)) were performed as described previously.

Poly(I—C) injection and hematopoietic cell counting: Poly(I—C) (Sigma) was dissolved in phosphate buffered saline and intraperitoneally (ip) injected to mice at 5 μg/g body weight (gbw). Blood and bone marrow nucleated cells were counted in Turk solution (3% acetic acid and 0.01% crystal violet in H₂O).

Bone marrow transplantation. Bone marrow cells (CD45.2+) were collected from UBP43+/+ and UBP43−/− donor mice five days after they were injected with 5-fluoruracil (5-FU) (Sigma) at 100 μg/gbw. C57/BL6 congenic strain C57/B6.SJL PEP3b-BoyJ (CD45.2−) recipient mice were lethally irradiated (1000 rads in a split dose separated by 4 hours) using a Gamma irradiator (J. L. Shepherd Model 143-45). One×10⁶ bone marrow cells from each individual donor mouse were injected intravenously into each irradiated recipient. Transplanted mice were kept in sterilized cages and fed with acidic water (pH 2.0) for three weeks before putting them back to regular care. The appearance of donor origin blood cells (CD45.2+) was detected by flow cytometric analysis with fluorescence isothiocyanate (FITC) conjugated CD45.2 antibodies (BD Biosciences).

In vitro bone marrow cell culture. The colony forming unit (CFU) assay was performed as described previously in the presence of the indicated concentration of IFNβ (Calbiochem) (Rhoades, Hetherington, Harakawa, Yergeau, Zhou, Liu, Little, Tenen, and Zhang, 2000). Liquid culture of bone marrow cells was performed in RPMI 1640 with 10% fetal bovine serum, 10 ng/ml IL-3 (peprotech), 10 ng/ml IL-6 (Peprotech), and 100 ng/ml SCF (Peprotech) in the presence or absence of 100 unit/ml IFNβ, 10 ng/ml IFNγ. (Peprotech), or 10 ng/ml TNFα (Calbiochem).

Apoptosis assay. The TUNEL assay was performed according to manufacturer's instructions (Boehringer Mannheim). A total of 200 cells were counted to determine the percentage of apoptotic cells. Annexin V-PE/7-AAD (7-amino-actinomycin D) apoptosis assay was performed using an apoptosis detection kit according to manufacturer's instructions (BD PharMingen).

Retroviral infection. UBP43 cDNA was subcloned into Bgl II and Hap I sites of MigR1. The production of replication defective retrovirus stock and bone marrow cell infection were conducted as previously described (Pear et al., Blood, 92: 3780 (1998).

Gel shift assay. Assays were performed as previously described (Malakhova et al., J. Biol. Chem., 277: 14703 (2002). Double stranded oligonucleotide from the ISG15 promoter that contains an ISGF3 binding site was used in the assay (Fu et al., Proc. Natl. Acad. Sci., 87: 8555 (1990).

Plasmid construction and transfection. UBE1L cDNA was kindly provided by Dr. Charles Buys (Kok et al., Proc. Natl. Acad. Sci., 90: 6071 (1993) and subcloned into pcDNA3 containing a 5′ end HA tag sequence generating pcDNA-HA-UBE1L. The UBP43 promoter-luciferase construct p3K-UBP43-luc was described previously (Malakhova et al., J. Biol. Chem., 277: 14703 (2002). Transfection of K562 cells was performed with electroporation (220 V, 975 μF). One×10⁷ K562 cells were transfected with p3K-UBP43-luc (4 μg), pcDNA3 or pcDNA-HA-UBE1L (6 μg), and promoterless Renilla luciferase construct pRL-luc (200 ng) as an internal control for transfection efficiency. Twenty-four hours after the electroporation, cells were split into two flasks and cultured in the absence or the presence of 1000 units/ml IFNα for the indicated length of time. Luciferase activities were analyzed using Dual-Luciferase assay system (Promega).

Western blotting. Antibodies against phospho-Stat1 Tyr701 (Cell Signaling), Stat1 (Santa Cruz), and HA (Babco) were purchased from the respective manufactures. Rabbit anti-mouse ISG15 polyclonal antibodies were generated using full length mouse ISG15. Rabbit polyclonal antibodies against human ISG15 were generously provided by Dr. E. Borden (D'Cunha et al., Proc. Natl. Acad. Sci., 93: 211 (1996). Western blotting was performed as previously described (Malakhov et al., J. Biol. Chem., 277: 9976 (2002).

Northern blotting. Total RNA was isolated using RNazol B reagent according to the manufacturer's instructions (TEL-TEST Inc.). Ten μg of total RNA from each sample were separated in an agarose/formaldehyde (0.22 M) gel, blotted on Hybond N⁺ membrane (Amersham), and probed with ³²p labeled cDNAs.

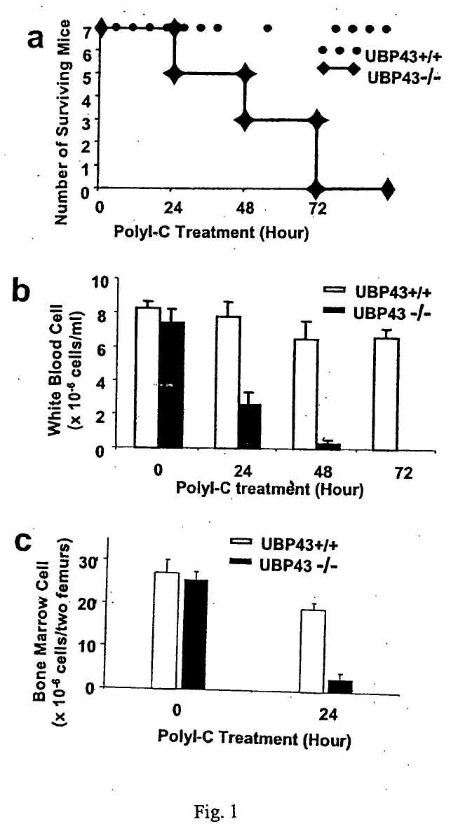

UBP43-null mice are sensitive to potent IFN-inducer poly(I—C). Injection with synthetic double stranded RNA-polyinosinic acid-polycytidylic acid (poly(I—C)) has been commonly used to induce endogenous IFN production in mice. (Kuhn et al., Science, 269: 1427 (1995). Therefore, poly(I—C) was used to assess the differences in response of UBP43−/− and UBP43+/+ mice. After daily poly(I—C) injections, UBP43+/+ mice (n=7) survived the course of treatment. Contrary to that, all UBP43−/− mice (n=7) died within 72 hours post treatment (FIG. 1a). The effect of poly (I—C) treatment on hematopoietic cells was also analyzed. As shown in FIG. 1b and FIG. 1c, UBP43+/+ mice responded to poly(I—C) by exhibiting a 20% decrease in number of total peripheral white blood cells and a 30% decrease in total bone marrow nucleated cells. A much stronger response was observed in UBP43−/− mice. There were dramatic decreases (over 90%) in the total number of white blood cells in peripheral blood and nucleated cells in bone marrow of UBP43−/− mice. These results indicate that UBP43 deficient mice are sensitive to poly(I—C).

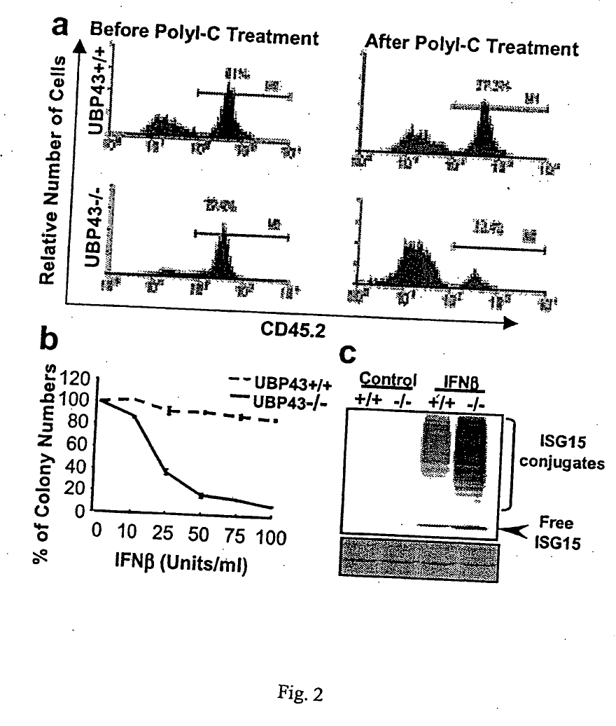

Sensitivity to IFN is intrinsic in UBP43-deficient hematopoietic cells. After birth, UBP43−/− mice gradually show defects associated with the central nervous system. To confirm that the hypersensitivity of hematopoietic cells to IFN is not due to other metabolic and physiological defects in UBP43−/− mice, bone marrow transplantation experiments were performed. UBP43 −/− mice were generated by crossing 129 and C57BU6 strains, and then backcrossing to the C57BL/6 strain. Therefore, their hematopoietic cells are CD45.2+. Lethally irradiated mice of C57/BL6 congenic strain C57/B6. SJL PEP3b-BoyJ were used as recipients in the bone marrow transplantation experiments to receive bone marrow cells from UBP43+/+ and UBP43−/− littermates. Hematopoietic cells of C57/B6. SJL PEP3b-BoyJ mice express CD45.1 on their cell surface, and can be distinguished from CD45.2 cells using specific antibodies in flow cytometric analysis. Four to six weeks after bone marrow transplantation, mice have generally recovered and manifest normal levels of hematopoietic cells. Bone marrow transplantation with cells from four UBP43+/+ mice and four UBP43−/− mice into individual C57/B6. SJL PEP3b-BoyJ mice was performed. These mice were healthy after recovery from lethal irradiation and bone marrow transplantation during a six month follow up period. Flow cytometric analysis showed that over 60% of hematopoietic cells were derived from either UBP43+/+ or UBP43−/− donor mice (FIG. 2a), indicating no significant difference in the ability of these bone marrow cells to engraft. The effect of poly (I—C) treatment on these mice was then tested. FIG. 2a shows a representative result from one mouse of each donor genotype. The relative amount of CD45.2+ cells contributed by UBP43+/+ donor in the peripheral blood did not change in response to poly(I—C); however, a significant decrease of CD45.2+ UBP43−/− cells was noted. Before dosing, 79% of blood cells were UBP43−/− cells, whereas 48 hours post administration, only 12% of blood cells were of the UBP43−/− genotype. These results demonstrate that the severe effect of poly(I—C) treatment is intrinsic to UBP43-deficient hematopoietic cells.

Besides activating type I IFN production, poly(I—C) also activates other signaling pathways related to viral infection (Kaufinan, Proc. Natl. Acad. Sci., 96: 11693 (1999)). To demonstrate that the hypersensitivity of UBP43−/− cells is directly related to type I IFN stimulation, in vitro bone marrow cell colony forming unit (CFU) assays were performed. The CFU assay is commonly used to study defects of bone marrow cells in response to various cytokines and growth factors. Type I IFN is known to suppress bone marrow cell proliferation and colony formation (Broxmeyer et al., J. Immunol., 131: 1300 (1983)). When equivalent numbers of UBP43+/+ and UBP43−/− bone marrow cells were cultured under regular CFU assay conditions in the absence or presence of 1000 units/ml IFNβ, UBP43+/+ bone marrow cells showed a 40% reduction in colony formation upon IFN stimulation while UBP43−/− bone marrow cells formed no colonies at all (data not shown). Such a difference in colony formation indicated an abnormal sensitivity of UBP43 deficient cells to IFNβ. Further, this sensitivity appeared to be dose-dependent: 50 units/ml of IFNβ resulted in over an 80% reduction of UBP43−/− bone marrow colony numbers (FIG. 2b) as well as a marked decrease in colony size (data not shown). Similar CFU assays using UBP43+/+ cells showed the appearance of regular sized colonies and less than a 10% reduction in colony number. These results demonstrate the sensitivity of UBP43−/− cells to type I IFN stimulation. Furthermore, consistent with UBP43 enzymatic specificity, a higher level of protein ISGylation is detected in UBP43−/− bone marrow cells relative to UBP43+/+ bone marrow cells (FIG. 2c).

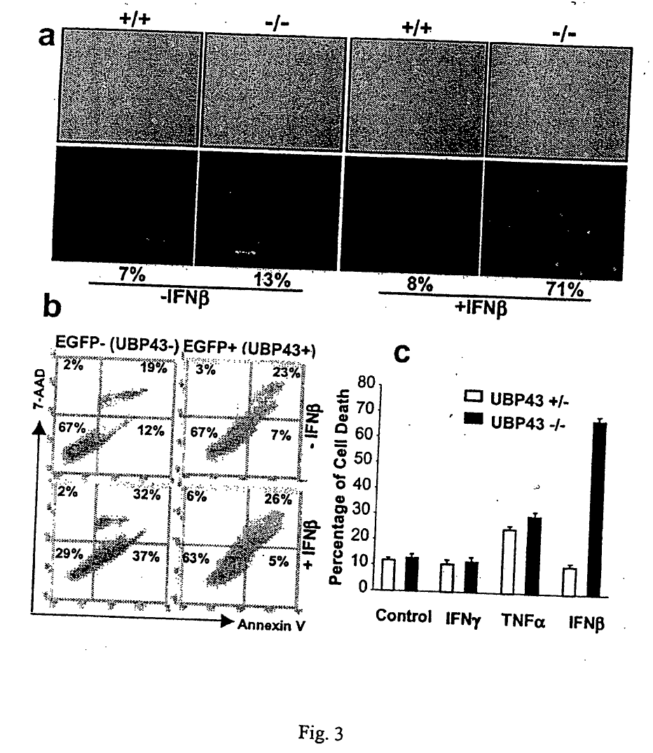

Augmented apoptosis of UBP43-deficient cells is specific to type I IFN stimulation. Previous studies have shown a reduced number of cells subsequent to IFN treatment (Stark et al., Annu. Rev. Biochem., 67: 227 (1998)). However, specific mechanisms through which IFN caused a reduction in cell number were not known. To determine whether the hypersensitivity of UBP43-deficient bone marrow cells to IFN is associated with augmented apoptosis, a tensional deoxyribosyl-transferase mediated dUTP nick end-labeling (TUNEL) assay was conducted on liquid bone marrow culture of UBP43+/+ and UBP43−/− cells following 48 hours culture in the absence or the presence of 100 units/ml of IFNβ. As presented in FIG. 3a, the percentage of apoptotic cells in UBP43+/+ bone marrow cell culture remained constant with the addition of IFNβ. However, a significant increase in number of apoptotic cell was observed in UBP43−/− bone marrow cells. To confirm that the observed increase of apoptosis in UBP43−/− cells was due to a lack of UBP43, UBP43 was reintroduced into UBP43−/− cells using a retroviral expression vector MigR1 (MSCV-IRES-EGFP) (Pear et al., Blood, 92: 3780 (1998)). Since UBP43 and EGFP were translated from the same transcript, EGFP+ cells also ectopically expressed UBP43. EGFP+ cells (UBP43 expressing) displayed significantly reduced apoptosis upon IFNβ treatment when compared to EGFP-cells (FIG. 3b). MigR1 vector alone infection did not rescue cells from IFN-induced apoptosis (data not shown). These results demonstrate that an augmented rate of apoptosis correlates with the hypersensitive response of UBP43 deficient cells to type I IFN.

In the next experiment, it was investigated whether other proapoptotic cytokines could have similar effect on UBP43−/− cells. TNFα and the type II IFN-IFNγ are commonly used to study bone marrow cell growth and apoptosis and in the analysis of various knockout mouse models. Therefore, liquid bone marrow cell culture assays to study the effect of TNFα, IFNγ, and IFNβ on UBP43± and UBP43−/− cells were performed. IFNβ treatment resulted in a substantial increase of apoptosis in UBP43−/− cells, whereas IFNγ and TNF-α did not cause a significant difference in cell death between UBP43± cells and UBP43−/− cells (FIG. 3c). This result indicates a specific role of type I IFN in the induction of apoptosis in UBP43-deficient cells.

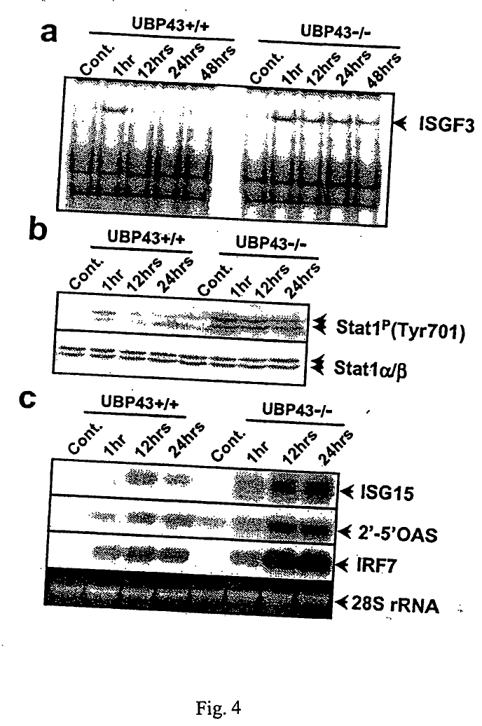

JAK-STAT signaling is extensively activated in UBP43-null cells upon IFN stimulation. The molecular basis for the action of IFN has not been understood despite tremendous past efforts. Thus, to analyze the IFN-hypersensitivity of UBP43 deficient cells, the increased protein ISGylation in these cells was investigated to determine if it alters type I IFN signaling. Accordingly, the DNA binding properties of the ISGF3 complex in gel shift assays was investigated. Using protein extracts prepared from UBP43+/+ and UBP43−/− bone marrow cells following stimulation with IFNβ for a maximum of 48 hours. In UBP43+/+ cells, ISGF3 DNA binding was rapidly and transiently induced upon addition of IFNβ and became undetectable by 12 hours of IFNβ stimulation (FIG. 4a). In contrast, strong ISGF3 DNA binding was still detectable in protein extracts from IFNβ treated UBP43−/− cells at 48 hours. Addition of antibodies against Stat1 and p48 also supershifted the ISGF3 complex. Consistent with this observation, prolonged Stat1 phosphorylation in UBP43−/− cells was identified by western blot using antibodies specifically recognizing tyrosine phosphorylated Stat1 (FIG. 4b). The expression pattern of IFN target genes, including ISG15, 2+-5′OAS and IRF7 (FIG. 4c) was also investigated. Upon IFβ stimulation of bone marrow cells, the expression of these genes was clearly activated in both UBP43+/+ and UBP43−/− cells. However, the level and the timing of activation were much higher and extended in UBP43−/− cells when compared with UBP43+/+ cells. In addition to exhibiting a higher level of protein ISGylation, the UBP43−/− cells also exhibited an increase of free ISG15 upon IFNβ stimulation when compared to controls (FIG. 2c). These results suggest that an augmented level of ISG15 conjugates and/or the absence of their turnover in UBP43 deficient cells may enhance and prolong the signaling of type I IFN.

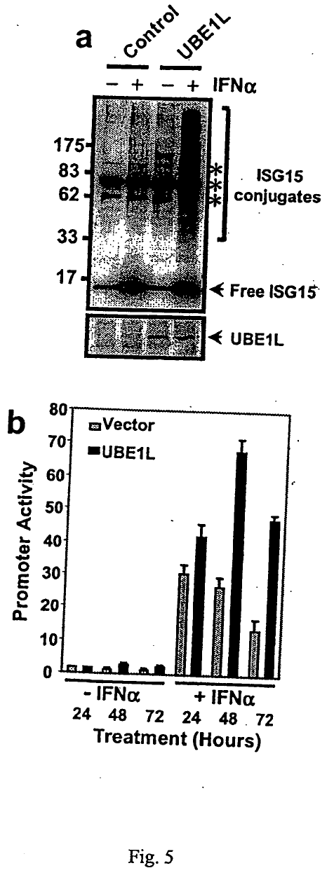

Protein ISGylation enhances IFN signaling. The effect of protein ISGylation on IFN signaling was examined in additional assays. K562 human leukemic cells do not have protein ISGylation upon type I IFN treatment (Loeb and Haas, J. Biol. Chem., 267: 7806 (1992)). UBE1L is an ISG15 activating enzyme (E1) that is crucial for ISG15 conjugation and was originally cloned from the study of chromosome 3p deletion associated with small cell lung cancer (Yuan and Krug, EMBO J., 20: 362 (2001); Kok et al., Proc. Natl. Acad. Sci., 90: 6071 (1993)). It was then investigated whether missing functional UBE1L was responsible for the lack of protein ISGylation in K562 cells. This was done by transiently transfecting HA-tagged UBE1L into K562 cells. After culturing cells in the presence or the absence of IFNα for 24 hours, protein extracts were subjected to Western blot analysis with anti-ISG15 antibodies. UBE1L expressing cells in contrast to controls showed a strong induction of protein ISGylation (FIG. 5a). This result demonstrates that lack of finctional UBE1L expression is the major reason for the absence of ISGylation upon IFN stimulation in K562 cells.

To further analyze whether protein ISGylation is involved in the enhanced and prolonged IFN signaling as observed in UBP43 deficient cells, an empty vector, or a UBE1L expression construct, was co-transfected into K562 cells with a luciferase reporter construct under the control of interferon-responsive elements (ISRE). The luciferase activity in the absence of exogenous UBE1L expression started to decline 24 hours post IFN stimulation (FIG. 5b). In the presence of exogenous UBE1L expression, significantly higher luciferase activity was detected at all three time points measured, reaching the maximum by 48 hours. These results further support the conclusion from the analysis in UBP43 deficient cells that protein ISGylation enhances and prolongs type I IFN signaling.

Two major cellular effects of IFN are the suppression of cell proliferation and the promotion of apoptosis (Stark et al., Annu. Rev. Biochem., 67: 227 (1998)). It is demonstrated herein for the first time that ISG15 protease UBP43 deficient cells are sensitive to type I IFN and undergo apoptosis upon IFN stimulation. Furthermore, enhanced and prolonged IFN signaling is detected in UBP43 deficient cells. Mechanisms responsible for the regulation of IFN signaling are known to operate at several levels, including the negative controls by down-regulation and degradation of receptors, regulation of JAKs and STATs by protein-tyrosine phosphatases (PTPs), sacs, and PIAS proteins (Greenhalgh and Hilton, J. Leukoc. Biol., 70: 348 (2001)). It has been demonstrated herein that UBP43 is a novel negative regulator of IFN signaling. Lack of UBP43 resulted in a profound increase in the level of protein ISGylation that is associated with the enhanced and prolonged type I IFN signaling. Furthermore, the role of ISGylation in the regulation of IFN signaling was also confirmed by transfection experiments in which reconstitution of ISG15 conjugation system in UBE1L-deficient K562 cells increases protein ISGylation and the activity of the interferon responsive promoter. These results show that UBP43 negatively regulates the IFN activated JAK-STAT signaling pathway by decreasing levels of protein ISGylation. As shown in FIG. 2c, many proteins can be modified by ISG15.

### Documents

Joklik, W. K. (1991) in Fundamental Virology (Field, B. N., ed), 2nd Ed., pp. 343-370, Raven Press, Ltd., New York

Kurzrock, R., Talpaz, M., and Gutterman, J. U. (1991) in Biologic Therapy of Cancer (Devita, V. T., Hellman, S., and Rosenberg, S. A., eds), pp. 247-274, J. B. Lippincott, Philadelphia.

Platanias, L. C., and Colamonici, A. R. (1992) J. Biol. Chem. 267, 24053-24057.

Platanias, L. C., Uddin, S., and Colamonici, A. R. (1994) J. Biol. Chem. 269, 17761-17764.

Muller, M., Briscoe, J., Laxton, C., Guschin, D., Ziemiecki, A., Silvennoinen, A., Harpur, A. G., Barbieri, G., Witthuhn, B. A., Schindler, C., Pelligrini, S., Wilks, A. F., Ihle, J. N., Stark, G. R., and Kerr, I. M. (1993) Nature 366, 129-135.

Muller, M., Laxton, C., Briscoe, J., Schindler, C., Improta, T., Darnell, J. E., Jr., Stark, G. R., and Kerr, I. M. (1993) EMBO J. 12, 4221-4228.

Velaquez, I., Fellows, M., Stark, G. R., and Pelligrini, S. (1994) Cell 70, 313-322

Levy, D. E., Kessler, D. S., Pine, R., and Darnell, J. E., Jr. (1989) Genes Dev. 3, 1362-1371.

Darnell, J. E., Jr., Kerr, I. M., and Stark, G. R. (1994) Science 264, 1415-1421.

Shuai, K. (1994) Curr. Opin. Cell. Biol. 6, 253-259.

Qureshi, S. A., Salditt-Georgieff, M., and Darnell, J. E., Jr.(1995) Proc. Natl. Acad. Sci. USA 92, 3829-3833.

Wilks, A. F., and Harpur, A. G. (1994) Bioessays 16, 313-320.

Estroz, Z., Kurzrock, R., and Talpaz, M. (1993) Interferons: Basic Principles and Clinical Applications, pp. 53-76, R. G. Landes Company, Texas

Schiller, J. H., Willson, J. K. H., Bittner, G., Wolberg, W. H., Hawkins, M. J., and Borden, E. C. (1986) J. Interferon Cytokine Res. 6, 615-625

Meyskens, F. L., Jr., Loescher, L., Moon, T. E., Takasugi, B., and Salmon, S. E. (1984) J. Clin. Oncol. 2, 1223-1228.

Colamonici, O., Domanski, P., Platanias, L. C., and Diaz, M. O. (1992) Blood 80, 744-749.

Heyman, M., Granders, D., Brondum-Nielsen, K., Cederblad, B., Liu, Y., Xu, B., and Einhom, S. (1994) Leukemia (Baltimore) 8, 425-434.

Billard, C., Sigaux, F., Castaigne, S., Valensi, F., Flandrin, G., Degos, L., Falcoff, E., and Aguet, M. (1986) Blood 67, 821-827.

Pfeffer, L. M., and Donner, D. B. (1990) Cancer Res. 50, 2654-2659.

Aman, P., and von Gabain, A. (1990) EMBO J. 9, 147-152.

Kanda, K., Decker, T., Aman, P., Wahlstrom, M., von Gabain, A., and Kallin, B. (1992) Mol. Cell. Biol. 12,4930-4936.

Dron, M., and Tovey, M. G. (1993) J. Interferon Cytokine Res. 13, 377-383

Xu, B., Grander, D., Sangfelt, O., and Einhom, S. (1994) Blood 84, 1942-1949.

Petricoin, E., III, David, M., Fang, H., Grimley, P., Lamer, A. C., and Vande Pol, S. (1994) Mol. Cell. Biol. 14, 1477-1486.

Johns, T. G., Mackay, I. R., Callister, K. A., Hertzog, P. J., Devenish, R. J., and Linnane, A. W. (1992) J. Natl. Cancer Inst. 84, 1185-1190.

Wines, B. D., Choe, C. C., Hatzinisiriou, I., Devenish, R. J., Linnane, A. W., and Ralph, S.J. (1993) Biochem. Mol. Biol. Int. 31, 1111-1120.

Hertzog, P. J., Johns, T. G., Callister, K. A., Dinatale, A., and Linnane, A. W. (1990) Biochem. Mol. Biol. Int. 22, 1095-1102

Uze, G., Lutfalla, G., and Gresser, I. (1990) Cell 60, 225-234.

Payne, M. J., Ralph, S. J., De Veer, M. J., Allen, K., Linnane, A. W., and Devenish, R. J. (1994) Biochem. Mol. Biol. Int. 33, 283-288.

Ralph, S. J., Wines, B. D., Payne, M. J., Grubb, D., Hatzinisiriou, I., and Devenish, R. J. (1995) J. Immunol. 154, 2248-2256.

Coates, A. S., and Crawford, M. (1977) J. Natl. Cancer Inst. 59, 1325-1329.

Whitehead, R. H., and Little, J. H. (1973) in Pigment Cell, Vol. 1 (McGovern, V. J., and Russell, P., eds), pp. 382-389, S. Karger, Basel, Switzerland

Luttrell, D. K., Lee, A., Lansing, T. J., Crosby, R. M., Jung, K. D., Willard, D., Luther, M., Rodriguez, M., Bennan, J., and Gilmer, T. M. (1994) Proc. Natl. Acad. Sci. USA 91, 83-87.

Sanchez, E. R., Meshinchi, S., Tienrungroj, W., Schlesinger, M. J., Toft, D. O., and Pratt, W. B. (1987) J Biol. Chem. 262, 6986-6991.

Gough, N. (1987) Anal. Biochem. 173, 93-95.

Sambrook, J., Fritsch, E. F., and Maniatis, T. (1982) Molecular Cloning: A Laboratory Manual, Cold Spring Harbor Laboratory, Cold Spring Harbor, N.Y.

Piechaczyk, M., Blanchard, J. M., Marty, L., Dani, C., Panabieres, F., Sabouty, S. E., Fort, P., and Jeanteur, P. (1984) Nucleic Acids Res. 12, 6951-6964.

Hassanain, H. H., Dai, W., and Gupta, S. L. (1993) Anal. Biochem. 213, 162-167.

Paulus, W., Baur, I., Boyce, M., Breakfield, X. O., and Reeves, S. A. (1996) J. Virol. 70, 62-67.

Pear, W. S., Nolan, G. P., Scott, M. L., and Baltimore, D. (1993) Proc. Natl. Acad. Sci. USA 90, 392-8396.

Jordan, M., Schallhom, A., and Wurm, F. M. (1996) Nucleic Acids Res. 24, 596-601.

Gao, P. Q., Sims, S. H., Chang, D. C., and Deisseroth, A. B. (1993) J. Biol. Chem. 268, 12380-12387.

Gutch, M. J., Daly, C., and Reich, N. C. (1992) Proc. Natl. Acad. Sci. USA 89, 11411-11415.

Schindler, C., Shuai, K., Prezioso, V. R., and Damell, J. E., Jr. (1992) Science 257, 809-815.

Improta, T., Pine, R., and Pfeffer, L. M. (1992) J. Interferon Cytokine Res. 12, 87-94

Eilers, A., Seegert, D., Schindler, C., Baccarini, M., and Decker, T. (1993) Mol. Cell. Biol. 13, 3245-3254.

Kolla, V., Lindner, D. J., Weihua, X., Borden, E. C., and Kalvakolanu, D. V. (1996) J. Biol. Chem. 271, 10508-10514.

Lengyel, P. (1993) Proc. Natl. Acad. Sci. USA 90, 5893-5895.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

I claim:

1. A method to identify an agent that alters UBP43 activity comprising:
(a) contacting a test cell with a candidate agent;
(b) determining the amount of an ISG-conjugate in the test cell;
(c) determining the amount of the ISG-conjugate in a control cell, wherein the control cell is not contacted with the candidate agent; and
(d) comparing the amount of ISG15-conjugate in the test cell to the amount of ISG15-conjugate in the control cell, wherein a difference in the amount of ISG15-conjugate indicates that the agent alters UBP43 activity.

2. The method of claim 1, wherein the test cell, the control cell, or both of the test cell and the control cell express an interferon receptor.

3. The method of claim 2 further comprising contacting the test cell, the control cell, or both the test cell and the control cell with IFNα, IFNβ, double-stranded ribonucleic acid, or lipopolysaccharide.

4. The method of claim 1, wherein the test cell is a UBP43^{+/+} cell, a UBP43^{±} cell, or a UBP43^{-/-} cell.

5. The method of claim 1, wherein the control cell is a UBP43^{+/+} cell, a UBP43^{±} cell, or a UBP43^{-/-} cell.

6. The method of claim 1, wherein the ISG15-conjugate is selected from the group consisting of ISG15-phospholipase Cγ1, ISG15-Jak1, ISG15-ERK1, ISG15-ERK2, ISG15-Stat1, and a variant thereof.

7. A method to identify an agent that alters UBP43 activity comprising:
(a) contacting a composition comprising UBP43 and a substrate, with a candidate agent, wherein the substrate comprises a donor molecule and an acceptor molecule;
(b) determining the fluorescence resonance energy transfer between the donor molecule and the acceptor molecule in step (a); and
(c) determining the fluorescence resonance energy transfer between the donor molecule and the acceptor molecule in the composition comprising UBP43 and the substrate, without the candidate agent, wherein a difference in the fluorescence resonance energy transfer between the donor molecule and the acceptor molecule indicates that the agent alters UBP43 activity.

8. The method of claim 7, wherein the substrate comprises an ISG15-conjugate.

9. The method of claim 8, wherein the ISG15-conjugate is selected from the group consisting of ISG15-phospholipase Cγ1, ISGI15-Jak1, ISG15-ERK1, ISG15-ERK2, ISG15-Stat1, and a variant thereof.

10. A method to identify an agent that inhibits UBP43 activity comprising:
(a) contacting a composition comprising UBP43, a substrate, with a candidate agent, wherein the substrate comprises a donor molecule and an acceptor molecule;
(b) determining the fluorescence resonance energy transfer between the donor molecule and the acceptor molecule in step (a); and
(c) determining the fluorescence resonance energy transfer between the donor molecule and the acceptor molecule in the composition comprising UBP43 and the substrate, without the candidate agent, wherein substantially no change in the fluorescence resonance energy transfer between the donor molecule and the acceptor molecule indicates that the agent inhibits UBP43 activity.

11. The method of claim 10, wherein the substrate comprises an ISG15-conjugate.

12. The method of claim 11, wherein the ISG15-conjugate is selected from the group consisting of ISG15-phospholipase Cγ1, ISG15-Jak1, ISG15-ERK1, ISG15-ERK2, ISG15-Stat1, and a variant thereof.

13. A method to induce a cell to undergo apoptosis comprising contacting a cell with a composition comprising an ISG15-conjugate, and measuring the presence or amount of cellular apoptosis, wherein the ISG15-conjugate is selected from the group consisting of ISG15-phospholipase Cγ1, ISG15-Jak1, ISG15-ERK1, ISG15-Stat1, and a variant thereof.

14. The method of claim 13, further comprising contacting the cell with IFNα, IFNβ, double stranded ribonucleic acid, or lipopolysaccharide.

15. The method of claim 13, wherein the ISG15-conjugate cannot be cleaved by UBP43.

16. The method of claim 13, wherein the ISG15-conjugate includes a non-hydrolysable bond.

17. The method of claim 13, wherein the composition further comprises a pharmaceutically acceptable carrier.

18. The method of claim 13, wherein the composition is contained within a liposome.

19. A method to prolong or increase the response of a cell to interferon comprising contacting the cell with a composition comprising an ISG15-conjugate, contacting the cell with an interferon, and measuring the response of the cell to the interferon, wherein the ISG15-conjugate is selected from the group consisting of ISG15-phospholipase Cγ1, ISG15-Jak1, ISG15-ERK1, ISG15-Stat1, and a variant thereof.

20. The method of claim 19, wherein the ISG15-conjugate cannot be cleared by UBP43.

21. The method of claim 19, wherein the composition further comprises a pharmaceutically acceptable carrier.

22. The method of claim 19, wherein the composition is contained within a liposome.

23. The method of claim 19, further comprising contacting the cell with IFNα, IFNβ, double-stranded ribonucleic acid, or lipopolysaccharide.

24. A method to inhibit cell proliferation comprising, contacting a cell with a composition comprising an ISG15-conjugate, wherein the ISG15-conjugate is selected from the group consisting of ISG15-phospholipase Cγ1, ISG15-Jak1, ISG15-ERK1, ISG15-Stat1, and a variant thereof.

25. The method of claim 24, wherein the composition further comprises a pharmaceutically acceptable carrier.

26. The method of claim 24, wherein the composition is contained within a liposome.

27. The method of claim 24, further comprising contacting the cell with an interferon, double-stranded ribonucleic acid, or lipopolysaccharide.

28. The method of claim 24, wherein the cell is a tumor cell.

29. A method to inhibit the infection or replication of a virus comprising contacting a cell exposed to the virus with a composition comprising an ISG15-conjugate, wherein the ISG15-conjugate is selected from the group consisting of ISG15-phospholipase Cγ1, ISG15-Jak1, ISG15-ERK1, ISG15-Stat1, and a variant thereof.

30. The method of claim 29, further comprising contacting the cell with an interferon, double-stranded ribonucleic acid, or lipopolysaccharide.

31. The method of claim 29, wherein the virus is selected from the group consisting of hepatitis B virus, hepatitis C virus, human immunodeficiency virus, Epstein-Barr virus, herpes virus, rhinovirus, picomavirus, lentivirus, cytomegalovirus, respiratory syncytial virus, and poxvirus.

32. A method to treat an autoimmune disease comprising administering to a patient in need thereof an effective amount of a composition comprising an ISG15-conjugate, wherein the ISG15-conjugate is selected from the group consisting of ISG15-phospholipase Cγ1, ISG15-Jak1, ISG15-ERK1, ISG15-ERK2, ISG15-Stat1, and a variant thereof.

33. The method of claim 35, wherein the autoimmune disease is multiple sclerosis.

34. The method of claim 35, further comprising administering to the patient an IFNα, IFNβ, double stranded ribonucleic acid, or lipopolysaccharide.

35. A method to increase the effectiveness of an anticancer therapeutic comprising administering to a patient in need thereof an effective amount of the anticancer therapeutic, and administering to the patient an effective amount of a composition comprising an ISG15-conjugate, wherein the ISG15-conjugate is selected from the group consisting of ISG15-phospholipase Cγ1, ISG15-Jak1, ISG15-ERK1, ISG15-ERK2, ISG15-Stat1, and a variant thereof.

36. The method of claim 32, wherein the anticancer therapeutic is a cancer vaccine.

37. The method of claim 32, further comprising administering to the patient an INFα, IFNβ, double-stranded ribonucleic acid, or lipopolysaccharide.

38. An ISG15-conjugate that cannot be cleaved by UBP43.

39. The ISG15-conjugate of claim 38, wherein the ISG15-conjugate includes a non-hydrolysable bond at the cleavage site used by UBP43.

40. The method of claim 38, wherein the ISG15-conjugate is selected from the group consisting of ISG15-phospholipase Cγ1, ISG15-Jak1, ISG15-ERK1, ISG15-ERK2, ISG15-Stat1, and a variant thereof.

41. A composition comprising an ISG15-conjugate contained within a liposome and a pharmaceutically acceptable carrier.

42. The composition of claim 41, wherein the ISG15-conjugate is selected from the group consisting of ISG15-phospholipase Cγ1, ISG15-Jak1, ISG15-ERK1, ISG15-ERK2, ISG15-Stat1, and a variant thereof.

43. The composition of claim 41 further comprising interferon, double-stranded ribonucleic acid, or lipopolysaccharide.

44. A method to increase phagocytic activity of a cell comprising contacting a cell with a composition comprising an ISG15-conjugate.

45. A method to increase cell motility comprising contacting a cell with a composition comprising an ISG15-conjugate.

46. A method to enhance wound healing comprising contacting the wound with a composition comprising an ISG15-conjugate.

47. A method to promote or increase apoptosis in a cell comprising contacting the cell with a composition comprising an agent that inhibits UBP43 activity.

48. A method to prolong or increase the response of a cell to interferon comprising contacting the cell with a composition comprising an agent that inhibits UBP43 activity, contacting the cell with an interferon.

49. A method to inhibit the infection or replication of a virus comprising contacting a cell exposed to the virus with a composition comprising an agent that inhibits UBP43 activity.

50. A method to treat an autoimmune disease comprising administering to a patient in need thereof an effective amount of a composition comprising an agent that inhibits UBP43 activity.

51. A method to increase the effectiveness of an anticancer therapeutic comprising administering to a patient in need thereof an effective amount of the anticancer therapeutic, and administering to the patient an effective amount of a composition comprising an agent that inhibits UBP43 activity.

52. A method to increase phagocytic activity of a cell comprising contacting a cell with a composition comprising an agent that inhibits UBP43 activity.

53. A method to increase cell motility comprising contacting a cell with a composition comprising an agent that inhibits UBP43 activity.

54. A method to enhance wound healing comprising contacting the wound with a composition comprising an agent that inhibits UBP43 activity.
